(19) United States
(12) Patent Application Publication
(10) Pub. No.: US 2024/0166596 A1
(43) Pub. Date: May 23, 2024

(54) SULFAMOYL BENZENE DERIVATIVES AND USES THEREOF

(71) Applicant: AphioTx Inc., Calgary (CA)

(72) Inventors: Tuan TRANG, Calgary (CA); Darren Jason DERKSEN, Cochrane (CA); Kathleen NAVIS, Sherwood Park (CA); Evgueni GOROBETS, Calgary (CA); James PAPATZIMAS, Calgary (CA)

(21) Appl. No.: 18/024,443

(22) PCT Filed: Jul. 15, 2022
(86) PCT No.: PCT/CA2022/051102

§ 371 Date: Mar. 2, 2023

Related U.S. Application Data
(60) Provisional application No. 63/222,704, filed on Jul. 16, 2021.

(51) Int. Cl. C07C 317/32; A61P 25/36; C07D 211/56; C07D 231/12; C07D 231/56; C07D 239/42

(52) U.S. Cl. CPC C07C 317/32; A61P 25/36; C07D 211/56; C07D 231/12; C07D 231/56; C07D 239/42

(57) ABSTRACT

The present invention provides compounds of formula (I): NR1R2SOOYR3 (I) or a pharmaceutically acceptable salt thereof. The present invention also provides pharmaceutical compositions comprising a compound of formula (I), and use of the com-pounds and the pharmaceutical compositions for treating or ameliorating opioid withdrawal syndrome in a subject.

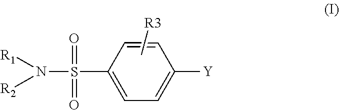

### FIELD OF THE INVENTION

The present invention pertains to the field of treatment of substance-use disorders pertaining to opioids. More particularly, the present disclosure relates to novel sulfamoyl benzene derivatives for ameliorating one or more opioid withdrawal symptoms and ameliorating opioid withdrawal syndrome.

### BACKGROUND OF THE INVENTION

Opioids provide potent pain relief and are used to treat a variety of chronic pain conditions. However, their long-term use can result in the development of physical dependence. Individuals that become physically dependent on opioids experience a debilitating withdrawal syndrome upon stopping opioid use. Symptoms of this withdrawal syndrome include gastrointestinal distress including abdominal cramping, nausea, diarrhea, stomach ache and vomiting; cardiovascular symptoms including high blood pressure and tachycardia, anxiety and depression. The severity of these symptoms is a major factor for continued opioid use, contributing to the socioeconomic burden of the global opioid epidemic.

Opioid seeking, drug craving and relapse are a significant hurdle to long term treatment of opioid drug addiction.

Converging evidence critically implicates microglia in both the physical and affective symptoms of opioid withdrawal. Microglia are immune cells in the central nervous system and key targets of opioid action. Pannexin-1 (Panx1) channels expressed on microglia have been implicated in opioid withdrawal. Pharmacological blockade of Panx1 using the Panx1 channel blocker probenecid ameliorated withdrawal behaviors in mice. Though probenecid is effective in attenuating withdrawal behaviors, it displays poor solubility, and has limited effectiveness at low dose. Therefore, there exists a need for new Panx1 channel blockers, which have high potency and are selective Panx1 inhibitors

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide novel sulfamoyl benzene derivatives.

In accordance with an aspect of the present invention, there is provided a compound having formula (I):

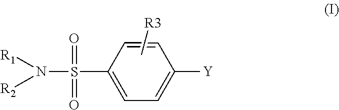

or a pharmaceutically acceptable salt thereof, wherein:

R1 and R2 are independently C₁-C₄ linear alkyl, or R1 and R2 are alkyl groups taken together with the N to form a 4 to 8 membered heterocyclic group;

R3 is H, or one or more substituents independently selected from —R, —OH, —OR, —X, —CX₃, —NR′R″, C₄-C₈ cyclic group or 5 to 7 membered heterocyclic group, wherein R is C₁-C₆ alkyl, X is F or Cl, R′ and R″ are independently H or C₁-C₆ alkyl;

Y is:
an unsaturated heterocyclic group having 1-3 N atoms (optionally substituted with one or more substituents selected from —R, —OR, —COOH, —COOR, —NR′R″, wherein R is C₁-C₆ alkyl, R′ and R″ are independently H or C₁-C₆ alkyl or R′ and R″ are alkyl groups taken together with a heteroatom to form a 5 to 7 membered heterocyclic group),
or
a group having the following formula:

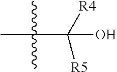

wherein R4 and R5 are independently selected from C₁-C₆ haloalkyl (wherein the halogen is independently selected from F or Cl), aryl or heterocycle.

In accordance with another aspect of the invention, there is provided a pharmaceutical composition comprising a compound of the invention as defined herein, and a pharmaceutically acceptable carrier.

In accordance with another aspect of the invention, there is provided a method of treating or ameliorating opioid withdrawal syndrome in a subject, comprising administering to the subject an effective amount of a compound or pharmaceutical composition of the invention.

In accordance with another aspect of the invention, there is provided a method of reducing risk of relapse to drug seeking in a subject, comprising administering to the subject an effective amount of a compound or pharmaceutical composition of the invention.

### BRIEF DESCRIPTION OF THE DRAWINGS

These and other features of the invention will become more apparent in the following detailed description in which reference is made to the appended drawings.

FIG. 1A illustrates Yo-Pro dye uptake measurements for Probenecid, and FIG. 1B illustrates Yo-Pro dye uptake measurements for exemplary Compound 1 in accordance with the present invention.

FIG. 2 illustrates Yo-Pro dye uptake measurements for exemplary Compounds 2 and 3, respectively, in accordance with the present invention.

FIGS. 3A, 3B and 3C, illustrate Yo-Pro dye uptake measurements for exemplary Compounds 4, 5 and 6, respectively, in accordance with the present invention.

FIG. 4 compares Panx1 inhibitory activity of probenecid and exemplary Compounds 1 and 5, respectively, in accordance with the present invention.

FIG. 5 illustrates that probenecid attenuates physical withdrawal behaviors in a naloxone-precipitated withdrawal model in mice. (a) Schematic depicting intraperitoneal drug administration paradigm in mice. Escalating doses of morphine (MS) were administered for 5 days. Probenecid (PRB) was given 1 hour after morphine on day 5, and withdrawal was precipitated 1 hour after that. Withdrawal behaviors were observed for half an hour following naloxone. (b-c) Quantification of composite withdrawal scores (One-way ANOVA with Dunnet post-hoc test (F4,34=52.28)) (b) and individual physical withdrawal behaviors (c) in C57BL/6J mice treated with saline (CTR n=8) or morphine (MS n=8) for 5 days, compared to mice that received 15, 25 or 50 mg/kg systemic probenecid (MS/PRB n=8 per treatment group). One-way ANOVA with Dunnet post-hoc for each behavior.

FIG. 6 illustrates that Compound 1 attenuates physical withdrawal behaviors in a naloxone-precipitated withdrawal model in mice. (a-b) Quantification of composite withdrawal scores (One-way ANOVA with Dunnet post-hoc test (F4,32=66.55)) (a) and individual physical withdrawal behaviors (b) in C57BL/6J mice treated with saline and 0.5 mg/kg Compound 1 (CTR n=4) or morphine (MS n=8) for 5 days, compared to mice that received morphine and 0.1, 0.5 or 1 mg/kg of systemic Compound 1 (MS/Compound 1 n=8 per treatment group). One-way ANOVA with Dunnet post-hoc for each behavior.

FIG. 7 illustrates that probenecid and Compound 1 are not effective at attenuating withdrawal behaviors when given 30 minutes before withdrawal. (a) Schematic depicting intraperitoneal drug administration paradigm in mice. Escalating doses of morphine (MS) were administered for 5 days. Probenecid (PRB) and Compound 1 (EG) were given 1.5 hours after morphine on day 5, and withdrawal was precipitated 30 minutes after that. Withdrawal behaviors were observed for half an hour following naloxone (b) Quantification of composite withdrawal scores in C57BLJ6J mice that received 50 mg/kg systemic probenecid (MS/PRB n=6) or 0.5 mg/kg of Compound 1 (MS/EG n=8). One-way ANOVA with Dunnet post-hoc for each behavior (F2, 19=0.8806).

FIG. 8 illustrates that Compound 1 reduces motivational opioid seeking behaviours. (A) Schematic representation of the time courses for morphine infusion, extinction and reinstatement in male and female Long Evans rats. (B) Morphine acquisition on fixed ratio (FR) schedules, where one, two, and five lever presses respectively results in delivery of morphine paired with light cues (1.5mg/kg, N=35). (C) Number of morphine infusions during morphine acquisition (N=35). (D) In the extinction phase where no light cue and morphine was given, all animals, including those receiving Compound 1 throughout extinction only (Compound 1+SAL) and those receiving Compound 1 throughout extinction and first day of reinstatement (Compound 1 +Compound 1) reduced lever pressing (morphine seeking behaviours), no difference was observed between animals receiving saline (SAL) of Compound 1. (E) In male animals, a trend in increasing morphine seeking behaviours was observed during the reinstatement phase (n=5), no significant differences were observed after Compound 1 treatments. (F) In female animals, SAL treatment increased morphine seeking behaviours during reinstatement phase (n=4), which is reversed by administration of Compound 1 during extinction (Compound 1+SAL, n=5), administration of Compound 1 on the first day of reinstatement (SAL +Compound 1, n=3) or administration of Compound 1 during both extinction and reinstatement (Compound 1+Compound 1, n=4). (G) All animals showed an increase in morphine seeking behaviours after saline treatment, rescued by Compound 1 either during extinction, first day of reinstatement, or during both extinction and reinstatement phases. Data presented as mean±SEM, *** p<0.001.

FIG. 9 illustrates that Conditioned Place Aversion (CPA) was reduced by i.p. administration of Compound 1 (0.5 mg/kg) in morphine-dependent mice. CPA scores between vehicle treated (N=13) and Compound 1 treated mice reveal a significant reduction in aversion to the naloxone-paired chamber (unpaired t-test, p=0.026, t=2.368, df=25). FIG. 9 illustrates CPA score at 1 day (FIG. 9A) and 7 days (FIG. 9B) after naloxone-precipitated withdrawal.

### DETAILED DESCRIPTION OF THE INVENTION

Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The terms “patient,” “subject,” or “individual” are used interchangeably herein, and refer to any animal amenable to the methods described herein. In a non-limiting embodiment, the patient, subject or individual is a human.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “therapeutic” treatment is a treatment administered to a subject who exhibits signs or symptoms of pathology, disease, disorder, condition or illness, for the purpose of diminishing or eliminating those signs or symptoms.

As used herein, the term “treatment” or “treating” is defined as the application or administration of a therapeutic agent, i.e., a compound of the invention (alone or in combination with another pharmaceutical agent), to a patient, who has a condition contemplated herein, a sign or symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

As used herein, the terms “effective amount,” “pharmaceutically effective amount” and “therapeutically effective amount” refer to a nontoxic but sufficient amount of an agent to provide the desired biological result. That result may be reduction and/or alleviation of a sign, a symptom, or a cause of a disease or disorder, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, methanesulfonic (mesylate), hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

An “effective amount” of a delivery vehicle is that amount sufficient to effectively bind or deliver a compound.

As used herein, the term “halogen” refers to fluorine, bromine, chlorine, and iodine atoms unless otherwise specified.

As used herein, the term “alkyl” refers to a straight chain or branched alkyl group of one to ten carbon atoms unless otherwise specified. This term is further exemplified by such groups as methyl, ethyl, n-propyl, i-propyl, n-butyl, t-butyl, i-butyl, hexyl and the like.

As used herein, the term “heterocycle” refers to a carbocyclic group having a single ring (e.g., morpholino, pyridyl or furyl) or multiple condensed rings (e.g., naphthpyridyl, quinoxalyl, quinolinyl, indolizinyl, indanyl) and having at least one hetero atom, such as N O and S, within the ring. The rings may be independently saturated or unsaturated.

As used herein, the term “unsaturated heterocycle” includes “heteroaryl” group, which refers to a heterocycle in which at least one heterocyclic ring is aromatic. As used herein, the term “haloalkyl” refers to an alkyl group, wherein one or more hydrogen atoms are replaced by a halogen atom, such as −(CF₂)ₙCF₃, wherein n=0-5.

As used herein, the term “about” refers to approximately a +/−10% variation from a given value. It is to be understood that such a variation is always included in any given value provided herein, whether or not it is specifically referred to.

The present invention provides novel sulfamoyl benzene derivatives and their use for modulating opioid withdrawal symptoms and opioid withdrawal syndrome.

The present invention provides compounds of the general formula (I):

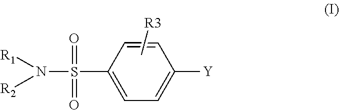

or a pharmaceutically acceptable salt thereof, wherein:
R1 and R2 are independently C₁-C₄ linear alkyl, or R1 and R2 are alkyl groups taken together with the N to form a 4 to 8 membered heterocyclic group;
R3 is H, or one or more substituents independently selected from —R, —OH, —OR, halogen, —CX₃, —NR′R″, C₄ -C₈ cyclic group or 5 to 7 membered heterocyclic group, wherein R is C₁-C₆ alkyl, X is F or Cl, R′ and R″ are independently H or C₁-C₆ alkyl;
Y is:
   an unsaturated heterocylic group having 1-3 N atoms (optionally substituted with one or more substituents selected from —R, —OR, —COOH, —COOR, —NR′R″, wherein R is C₁-C₆ alkyl, R′ and R″ are independently H or C₁-C₆ alkyl or R′ and R″ are alkyl groups taken together with a heteroatom to form a 5 to 7 membered heterocyclic group),
   or
   a group having the following formula:

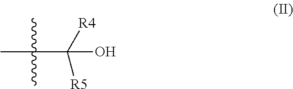

wherein R4 and R5 are independently selected from C₁-C₆ haloalkyl (wherein the halogen is independently selected from F or Cl), aryl or heterocycle.

In some embodiments, for R3 halogen is F or Cl.

In some embodiments, in the compounds of formula (I), R1 and R2 are the same. In some embodiments, R4 and R5 are both C₁-C₆ alkyl.

In some embodiments, in the compound of formula (I), Y is an unsaturated heterocylic group having 1-3 N atoms (optionally substituted with one or more substituents selected from —R, —OR, —COOH, —COOR, —NR′R″, wherein R is C₁-C₆ alkyl, R′ and R″ are independently H or C₁-C₆ alkyl).

In some embodiments, in the compounds of formula (I), Y is:

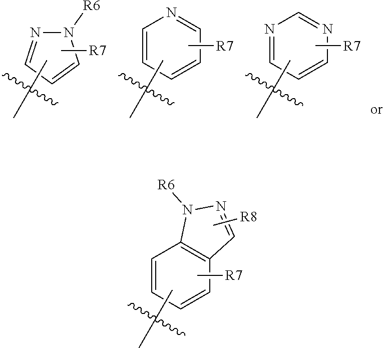

wherein:
R6 is H or alkyl,
R7 is H or one or more substituents independently selected from —R, —OR, —NR′R″, halogen or CN, 5 to 7 membered heterocyclic group, wherein R is C1-C6 alkyl, wherein R′ and R″ are independently selected from H or alkyl, or R′ and R″ taken together with the N form a 5-6 membered heterocycle, and

R8 is selected from R, NR′R″, wherein R is C1-C6 alkyl, R′ and R″ are independently H or C1-C6 alkyl.

In some embodiments, for R7 halogen is F or Cl.

In some embodiments, the compound of formula (I) is:

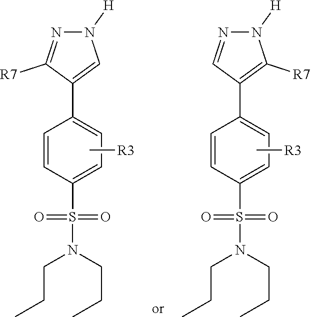

wherein: R3 and R7 are as defined above.

In some embodiments, in the compound of formula (I), Y is:

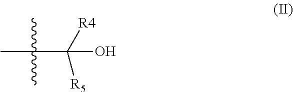

wherein R4 and R5 are independently selected from C₁-C₆ haloalkyl (wherein halogen is independently selected from F or Cl), aryl or heterocycle.

In some embodiments, in the structural formula (II), R4 and R5 are independently selected from C₁-C₆ haloalkyl. In some embodiments, R4 and R5 are same. In some embodiments, R4 and R5 are both —CF₃ or —CF₂CF₃.

In some embodiments, the compound of formula (I) includes the following exemplary compounds:

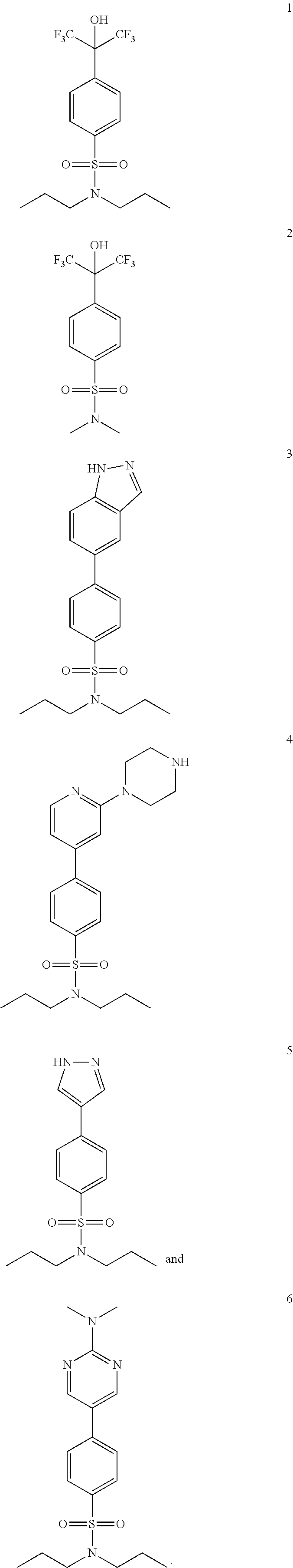

In some embodiments, the compound of formula (I) includes the following exemplary compounds:

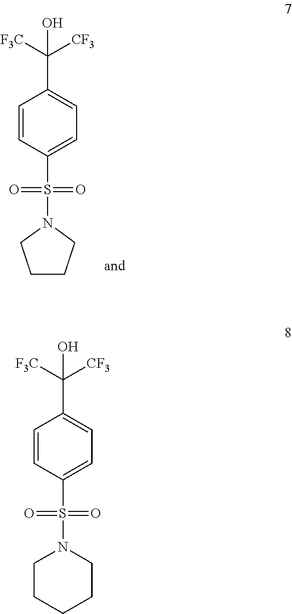 [SMILES: O=S(=O)(c1ccc(C(O)(C(F)(F)F)C(F)(F)F)cc1)N1CCCC1 ; O=S(=O)(c1ccc(C(O)(C(F)(F)F)C(F)(F)F)cc1)N1CCCCC1]

To gain a better understanding of the invention described herein, the following examples are set forth with reference to the accompanying drawings. It will be understood that these examples are intended to describe illustrative embodiments of the invention and are not intended to limit the scope of the invention in any way.

### Preparation of Compounds of Formula (I)

In an aspect, the present invention provides processes/methods for preparing compounds of formula (I).

In one embodiment, the compounds of formula (I), wherein Y is an unsaturated heterocycle, can be prepared via cross coupling reaction between a compound of formula (III), wherein Z is halogen or —OTf (i.e CF₃SO₃—) or —OAc, and an organometallic compound, in the presence of a suitable metal catalyst. Non limiting examples of the organometallic compounds include organoboron compounds (i.e. boronic acids, boronic esters), organozinc compounds, organosilanes, organomagnesium compounds (such as Grignard reagent), organonickel compounds and organocopper compounds. Non limiting examples of the catalysts for the cross coupling reaction include transition metal complexes, such as PdLn and NiLn, wherein L is a ligand such as triphenylphosphine, dppe, BINAP, or chiraphos.

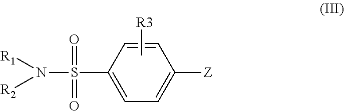

In one embodiment, the compounds of formula (I), wherein Y is an unsaturated heterocycle, can be prepared via a cross coupling reaction between a halide of formula (III) and a boronic ester of formula (IV) in the presence of a palladium or nickel catalyst in a suitable solvent:

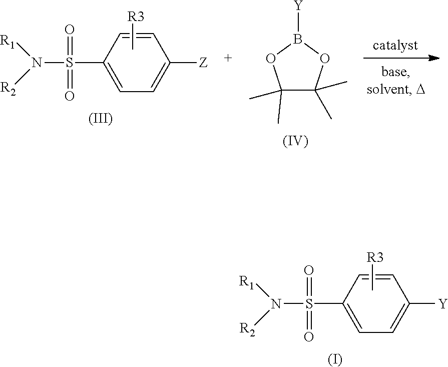

Non-limiting examples of palladium and nickel catalysts include PdLn and NiLn, respectively, wherein L is a ligand, such as triphenylphosphine, dppe, BINAP, or chiraphos. Non-limiting examples of suitable base include alkali metal carbonates (such as Na₂CO₃, K₂CO₃, Cs₂CO₃, etc.), alkali metal hydroxides (such as NaOH, KOH, etc.), or alkali metal acetates (such as NaOAc, KOAc, etc.), metal hydroxides (such as Ca(OH)₂), triethylamine (TEA), and t-butyl alcohol (TBA). Non-limiting examples of suitable solvents include MeCN:water, DMF, toluene, THF and dioxane.

In one embodiment, the compounds of formula (I), wherein Y is:

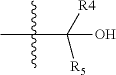

can be prepared by reacting an organometallic or a Grignard reagent formed from a halide compound of formula (III) with a ketone of formula (V):

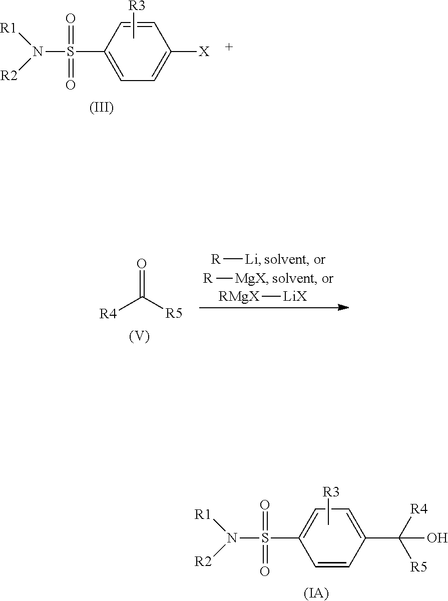

Non-limiting examples of R-Li include butyl-lithium, methyl-lithium, etc. Examples of R-MgX include iPrMgCl, iPrMgCl, MeMgCl, MeMgBr, PhMgBr, etc.

In one embodiment, the compounds of formula (I), wherein Y is:

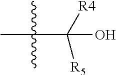

can be prepared by reacting a halide compound of formula (III) with a compound of formula (VI) to form a compound of formula (VII). Compound of formula (VII) is then reacted with a silane compound of formula (VIII) in the presence of a catalyst or an activating agent, and a fluoride source is a suitable solvent.

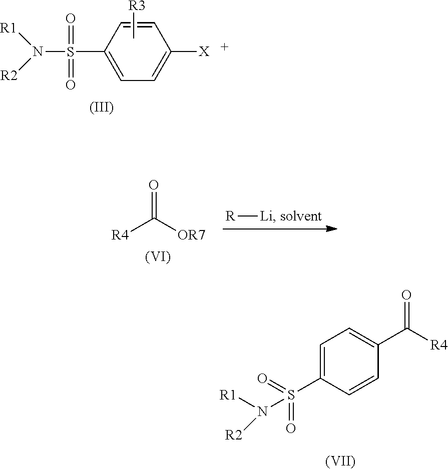

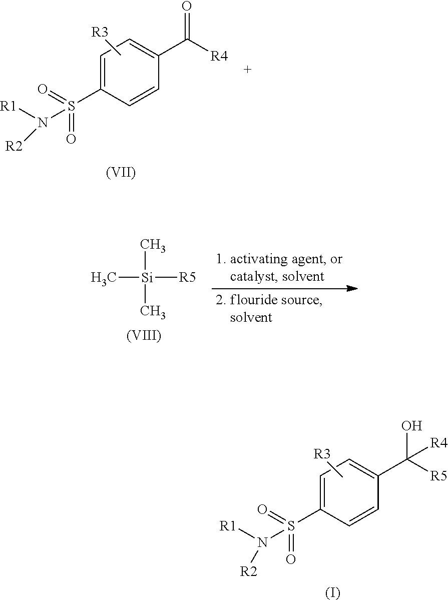

Non-limiting examples of R-Li include butyl-lithium, methyl-lithium, etc. Non-limiting examples of suitable solvents for the above reaction scheme include MeCN:water, DMF, toluene, THF and dioxane. Non limiting examples of suitable activating agent include CsF, and any strong nucleophile such as alkoxide. Non limiting examples of fluoride ion source include tetra-n-butylammonium fluoride (TBAF). Non limiting examples of catalyst for the reaction between compounds (VII) and (VIII) include CsF.

The compound of formula (III) can be prepared by amine alkylation reaction between a compound of formula (IX) and a compound of formula (X):

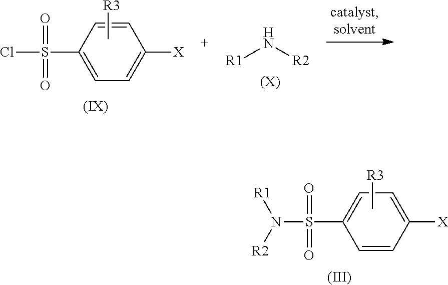

Non limiting examples of catalysts for the amine alkylation include DMAP etc., and the suitable solvents include dichloromethane, chloroform, etc.

The optimal reaction conditions and reaction time for each individual step may vary depending on the particular reagents used and substituents present in the reagents used. Unless otherwise indicated, one skilled in the art can readily select solvents, temperatures, and other reaction conditions. Exemplary procedures are presented in the Examples section. The reactions can be carried out using standard chemical processes and manipulations, for example, by removing the solvent from the residue and further purification in accordance with methods well known in the art, such as, without limitation, crystallization, distillation, extraction, grinding to powder and chromatography. Unless otherwise indicated, the starting materials are either commercially available or can usually be obtained from commercially available materials.

Routine experimentation, including appropriate manipulation of reaction conditions, reagents, and the synthetic procedure sequence, protecting any chemical functional group that is not compatible with the reaction conditions, and deprotecting at a suitable point in the sequence of process steps, are included in the scope of the invention. Suitable protecting groups and methods for protecting and deprotecting various substituents using such suitable protecting groups are well known to those skilled in the art; Examples that can be found in the user manual T. Greene and P. Wuts, Protecting Groups in Organic Synthesis (3^{rd ed.}) John Wiley & Sons, NY (1999), which is fully incorporated herein by reference. The synthesis of the compounds of the invention can be carried out by methods analogous to the methods in the synthesis schemes described above and in specific examples.

### Use of Compounds of Formula (I)

In one embodiment, the present invention provides a pharmaceutical composition for use in treating opioid withdrawal syndrome or at least one or more symptoms thereof in a subject wherein the composition comprises an effective amount of a compound of formula (I) in admixture with a suitable diluent or carrier.

According to one embodiment, the present invention provides a pharmaceutical composition for use in treating opioid withdrawal syndrome in a subject wherein the composition comprises an effective amount of a compound of formula (I).

According to one embodiment, the present invention provides a pharmaceutical composition for reducing risk of relapse to drug seeking wherein the composition comprises an effective amount of a compound of formula (I)

Such pharmaceutical compositions can be formulated for intralesional, intravenous, topical, rectal, parenteral, local, inhalant or subcutaneous, intradermal, intramuscular, intrathecal, transperitoneal, oral, and intracerebral use. The composition can be in a liquid, a solid, or a semisolid form, for example pills, tablets, creams, gelatin capsules, capsules, suppositories, soft gelatin capsules, gels, membranes, tubelets, solutions, or suspensions. Alternatively, the composition can be injected intravenously, intraperitoneally, or subcutaneously. Alternatively, the composition may comprise a topical delivery system exemplified by topical creams, lotions, emulsions, and transdermal patches.

The pharmaceutical compositions of the invention can be intended for administration to humans or animals. Dosages to be administered depend on individual needs, on the desired effect, and on the chosen route of administration.

Another embodiment of the present disclosure pertains to use of a compound of formula (I) to ameliorate opioid withdrawal syndrome by use of the methods and/or the compounds disclosed herein. Suitable dosing levels are readily determined by methods known in the art. Suitable dosing regimes are 8-h interval applications, twice daily applications, once daily applications, and therebetween. Alternatively, the dosing may be provided over extended periods of time via slow-release transdermal patches.

Another embodiment of the present disclosure generally relates to compositions comprising one or more of the compounds of the present invention. Suitable dosing levels are readily determined by methods known in the art. Suitable dosing regimes are 8-h interval applications, twice daily applications, once daily applications, and therebetween.

The pharmaceutical compositions comprising a compound of formula (I), disclosed herein can be prepared by known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

For example, pharmaceutical compositions of the disclosure comprising a compound of formula (I) disclosed herein, may be formulated for topical administration or alternatively, for transdermal administration to provide dosing over extended periods of time.

A pharmaceutical composition for topical administration may be provided as, for example, ointments, creams, suspensions, lotions, powders, solutions, pastes, gels, hydrogels, sprays, aerosols, dressings, or oils. When formulated in an ointment, the active ingredient may be employed with either a paraffmic or a water-miscible ointment base. Alternatively, the active ingredient may be formulated in a cream with an oil-in-water base or a water-in-oil base. Other formulations the compositions can be incorporated into include oils, suppositories, foams, liniments, aerosols, buccals, and sublingual tablets or topical devices for absorption through the skin or mucous membranes.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays are conveniently delivered from pressurized packs, for example, via a specially shaped closure. Oil-In-Water emulsions can also be utilized in the compositions, patches, bandages and articles. These systems are semisolid emulsions, micro-emulsions, or foam emulsion systems. Usually such a system has a “creamy white” appearance. The oleaginous phase may contain, but is not limited to, long-chain alcohols (cetyl, stearyl), long-chain esters (myristates, palmitates, stearates), long-chain acids (palmitic, stearic), vegetable and animal oils and assorted waxes. These can be made with anionic, cationic, nonionic or amphoteric surfactants, or with combinations especially of the nonionic surfactants. A typical invention gel base, provided herein for exemplary purposes only, can contain lecithin, isopropyl palmitate, poloxamer 407, and water. Topical carriers with different viscosities and hand-feel are known to the art. The above active ingredients can be dispersed within the pharmaceutically acceptable carrier in therapeutically effective amounts to treat neuropathies, and the other maladies described above.

A pharmaceutical composition for transdermal administration may be provided as, for example, a hydrogel comprising agents as described herein incorporated into an adhesive patch composition intended to remain in intimate contact with a subject's epidermis for a prolonged period of time. An exemplary adhesive patch composition can comprise a monolithic layer produced by mixing a compound of formula (I) with a silicone-type adhesive or alternatively an acrylate-vinyl acetate adhesive in a solvent exemplified by methylene chloride, ethyl acetate, isopropyl myristate, and propylene glycol. The mixture would then be extruded onto a polyester-backing film to a uniform thickness of about 100 microns or greater with a precision wet-film applicator. The solvent is allowed to evaporate in a drying oven and the resulting “patch” is trimmed to the appropriate size.

The pharmaceutical for topical administration or alternatively for transdermal administration of a compound of formula (I) may additionally incorporate a penetration enhancer and/or a thickening agent or gelling agent and/or an emollient and/or an antioxidant and/or an antimicrobial preservative and/or an emulsifying agent and/or a water miscible solvent and/or an alcohol and/or water.

According to one aspect, the pharmaceutical composition for topical administration or transdermal administration a compound of formula (I) may comprise one or more penetration enhancing agent or co-solvent for transdermal or topical delivery. A penetration enhancer is an excipient that aids in the diffusion of the active through the stratum corneum. Many penetration enhancers also function as co-solvents which are thought to increase the thermodynamic activity or solubility of a compound of formula (I). Penetration enhancers are also known as accelerants, adjuvants or sorption promoters. A suitable penetration enhancer for use in the pharmaceutical compositions and methods described herein should: (i) be highly potent, with a specific mechanism of action; (ii) exhibit a rapid onset upon administration; (iii) have a predictable duration of action; (iv) have only non-permanent or reversible effects on the skin; (v) be chemically stable; (vi) have no or minimal pharmacological effects; (vii) be physically and chemically compatible with other composition components; (viii) be odorless; (ix) be colorless; (x) be hypoallergenic; (xi) be non-irritating; (xii) be non-phototoxic; (xiii) be non-comedogenic; (xiv) have a solubility parameter approximating that of the skin (10.5 cal/cm³); (xv) be readily available; (xvi) be inexpensive; and (xvii) be able to formulated in pharmaceutical compositions for topical or transdermal delivery of an active pharmaceutical agent.

Several classes of chemical compounds, with various mechanisms of action, can be used as penetration enhancers. Set forth below are non-limiting examples of penetration enhancing agents, many of which are also suitable co-solvents. Sulfoxides, such as dimethylsulfoxide and decylmethylsulfoxide can be used as penetration enhancing agents. Dimethylsulfoxide enhances penetration in part by increasing lipid fluidity and promoting drug partitioning. In contrast, decylmethylsulfoxide enhances penetration by reacting with proteins in the skin that change the conformation of the proteins, which results in the creation of aqueous channels.

Another class of penetration enhancers are alkanones, such as N-heptane, N-octane, N-nonane, N-decane, N-undecane, N-dodecane, N-tridecane, N-tetradecane and N-hexadecane. Alkanones are thought to enhance the penetration of an active agent by altering the stratum corneum. A further class of penetration enhancers are alkanol alcohols, such as ethanol, propanol, butanol, 2-butanol, pentanol, 2-pentanol, hexanol, octanol. nonanol, decanol and benzyl alcohol. Low molecular weight alkanol alcohols, i.e., those with 6 or less carbons, may enhance penetration in part by acting as solubilizing agents, while more hydrophobic alcohols may increase diffusion by extracting lipids from the stratum corneum. A further class of penetration enhancers are fatty alcohols, such as oeyl alcohol, caprylic alcohol, decyl alcohol, lauryl alcohol, 2-lauryl alcohol, myristyl alcohol, cetyl alcohol, stearyl alcohol, oeyl alcohol, linoleyl alcohol and linolenyl alcohol. Polyols, including propylene glycol, polyethylene glycol, ethylene glycol, diethylene glycol, triethylene glycol, dipropylene glycol, glycerol, propanediol, butanediol, pentanediol, hexanetriol, propylene glycol monolaurate and diethylene glycol monomethyl ether (transcutol), can also enhance penetration. Some polyols, such as propylene glycol, may function as a penetration enhancer by solvating alpha-kertin and occupying hydrogen bonding sites, thereby reducing the amount of active-tissue binding.

Another class of penetration enhancers are amides, including urea, dimethylacetamide, diethyltoluamide, dimethylformamide, dimethyloctamide, dimethyldecamide and biodegradable cyclic urea (e.g., 1-alkyl-4-imidazolin-2-one). Amides have various mechanisms of enhancing penetration. For example, some amides, such as urea increase the hydration of the stratum corneum, act as a keratolytic and create hydrophilic diffusion channels. In contrast, other amides, such as dimethylacetamide and dimethylformamide, increase the partition to keratin at low concentrations, while increasing lipid fluidity and disrupting lipid packaging at higher concentrations. Another class of penetration enhancing agents are pyrrolidone derivatives, such as 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-methyl-pyrrolidone, N-cyclohexylpyrrolidone, N-dimethylaminopropyl-pyrrolidone, N-cocoalkypyrrolidone and N-tallowalkypyrrolidone, as well as biodegradable pyrrolidone derivatives, including fatty acid esters of N-(2-hydroxyethyl)-2-pyrrolidone. In part, pyrrolidone derivatives enhance penetration through interactions with the keratin in the stratum corneum and lipids in the skin structure. An additional class of penetration enhancers are cyclic amides, including I-dodecylazacycloheptane-2-one also known as AZONE® (AZONE is a registered trademark of Echo Therapuetics Inc., Philadelphia, Pa., USA), 1-geranylazacycloheptan-2-one, 1-farnesylazacycloheptan-2-one, 1-geranylgeranylazacycloheptan-2-one, 1-(3,7-dimethyloctyI)-azacycloheptan-2-one, 1-(3, 7, 1 1-trimefhyldodecyl)azacyclohaptan-2-one, 1-geranylazacyclohexane-2-one, 1-geranylazacyclopentan-2,5-dione and I-famesylazacyclopentan-2-one. Cyclic amides, such as AZONE®, enhance the penetration of active agents in part by affecting the stratum corneum's lipid structure, increasing partitioning and increasing membrane fluidity.

Additional classes of penetration enhancers include diethanolamine, triethanolamine and hexamethylenlauramide and its derivatives.

Additional penetration enhancers include linear fatty acids, such as octanoic acid, linoleic acid, valeric acid, heptanoic acid, pelagonic acid, caproic acid, capric acid, lauric acid, myristric acid, stearic acid, oleic acid and caprylic acid. Linear fatty acids enhance penetration in part via selective perturbation of the intercellular lipid bilayers. In addition, some linear fatty acids, such as oleic acid, enhance penetration by decreasing the phase transition temperatures of the lipid, thereby increasing motional freedom or fluidity of the lipids. Branched fatty acids, including isovaleric acid, neopentanoic acid, neoheptanoic acid, nonanoic acid, trimethyl hexaonic acid, neodecanoic acid and isostearic acid, are a further class of penetration enhancers. An additional class of penetration enhancers are aliphatic fatty acid esters, such as ethyl oleate, isopropyl n-butyrate, isopropyl n-hexanoate, isopropyl n-decanoate, isopropyl myristate (“IPM”), isopropyl palmitate and octyldodecyl myristate. Aliphatic fatty acid esters enhance penetration by increasing diffusivity in the stratum corneum and/or the partition coefficient. In addition, certain aliphatic fatty acid esters, such as IPM, enhance penetration by directly acting on the stratum corneum and permeating into the liposome bilayers thereby increasing fluidity. Alkyl fatty acid esters, such as ethyl acetate, butyl acetate, methyl acetate, methyl valerate, methyl propionate, diethyl sebacate, ethyl oleate, butyl stearate and methyl laurate, can act as penetration enhancers. Alkyl fatty acid esters enhance penetration in part by increasing the lipid fluidity.

An additional class of penetration enhancers are anionic surfactants, including sodium laurate, sodium lauryl sulfate and sodium octyl sulfate. Anionic surfactants enhance penetration of active agents by altering the barrier function of the stratum corneum and allowing removal of water-soluble agents that normally act as plasticizers. A further class of penetration enhancers are cationic surfactants, such as cetyltrimethylammonium bromide, tetradecyltrimethylammonium, octyltrimethyl ammonium bromide, benzalkonium chloride, octadecyltrimethylammonium chloride, cetylpyridinium chloride, dodecyltrimethylammonium chloride and hexadecyltrimethylammonium chloride. Cationic surfactants enhance penetration by adsorbing at, and interacting with, interfaces of biological membranes, resulting in skin damage. A further class of penetration enhancers are zwitterionic surfactants, such as hexadecyl trimethyl ammoniopropane sulfonate, oeyl betaine, cocamidopropyl hydroxysultaine and cocamidopropyl betaine. Nonionic surfactants exemplified by Polyxamer 231, Polyxamer 182, Polyxamer 184, Polysorbate 20, Polysorbate 60, BRIJ® 30, BRIJ® 93, BRIJ® 96, BRIJ® 99 (BRIJ is a registered trademark of Brij Image & Information Inc., Greensboro, N.C., USA), SPAN® 20, SPAN® 40, SPAN® 60, SPAN® 80, SPAN® 85 (SPAN is a registered trademark of Croda International PLC, East Yorkshire, UK), TWEEN® 20, TWEEN® 40, TWEEN® 60, TWEEN® 80 (TWEEN is a registered trademark of Uniqema Americas LLC, Wilmington, Del., USA), Myrj 45, MYRJ® 51, MYRJ® (MYRJ is a registered trademark of Uniqema Americas LLC, Wilmington, Del., USA), and MIGLYOL® 840 (MIGLYOL is a registered trademark of Cremer Oleo GMBH & Co., Hamburg, Fed. Rep. Germany), and the like. Nonionic surfactants enhance penetration in part by emulsifying the sebum and enhancing the thermodynamic activity or solubility of the active.

Another class of penetration enhancer increase the thermodynamic activity or solubility of the active, which include, but are not limited to, n-octanol, sodium oleate, D-limonene, monoolein, cineol, oeyl oleate, and isopropryl myristate.

Other penetration enhancers are bile salts, such as sodium cholate, sodium salts of taurocholic acid, glycolic acids and desoxycholic acids. Lecithin also has been found to have penetration enhancing characteristics. An additional class of penetration enhancers are terpenes, which include hydrocarbons, such as d-limonene, alpha-pinene and beta-carene; alcohols, such as, alpha-terpineol, terpinen-4-ol and carvol; ketones, such ascarvone, pulegone, piperitone and menthone; oxides, such as cyclohexene oxide, limonene oxide, alpha-pinene oxide, cyclopentene oxide and 1,8-cineole; and oils such as ylang ylang, anise, chenopodium and eucalyptus. Terpenes enhance penetration in part by disrupting the intercellular lipid bilayer to increase diffusivity of the active and opening polar pathways within and across the stratum corneum. Organic acids, such as salicylic acid and salicylates (including their methyl, ethyl and propyl glycol derivates), citric acid and succinic acid, are penetration enhancers. Another class of penetration enhancers are cyclodextrins, including 2-hydroxypropyl-beta-cyclodextrin and 2,6-dimethyl-beta-cyclodextrin. Cyclodextrins enhance the permeation of active agents by forming inclusion complexes with lipophilic actives and increasing their solubility in aqueous solutions.

The penetration enhancing agent(s) and/or co-solvent(s) is/are present in the pharmaceutical composition for topical administration or transdermal administration of a compound of formula (I) in an amount sufficient to provide the desired level of drug transport through the stratum corneum and epidermis or to increase the thermodynamic activity or solubility of the compound of formula (I). The one or more pharmaceutically acceptable penetration enhancer and/or co-solvent may be present in a total amount by weight of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1. 0.5%, about 1. 0.6%, about 1.7%, about 1.8%, about 1.9%, about 2.0%, about 2.1%, about 2. 0.2%, about 2. 0.3%, about 2.4%, about 2.5%, about 2.6%, about 2.7%, about 2.8%, about 2, 0.9%, about 3. 0.0%, about 3.1%, about 3.2%, about 3.3%, about 3.4%, about 3.5%, about 3 0.6%, about 3 0.7%, about 3.8%, about 3.9%, about 4.0%, about 4.1%, about 4.2%, about 4. 0.3%, about 4 0.4%, about 4.5%, about 4.6%, about 4.7%, about 4.8%, about 4.9%, about 5. 0.0%, about 5 0.1%, about 5.2%, about 5.3%, about 5.4%, about 5.5%, about 5.6%, about 5. 0.7%, about 5, 0.8%, about 5.9%, about 6.0%, about 6.1%, about 6.2%, about 6.3%, about 6. 0.4%, about 6, 0.5%, about 6.6%, about 6.7%, about 6.8%, about 6.9%, about 7.0%, about 7. 0.1%, about 7 0.2%, about 7.3%, about 7.4%, about 7.5%, about 7.6%, about 7.7%, about 7. 0.8%, about 7. 0.9%, about 8.0%, about 8.1%, about 8.2%, about 8.3%, about 8.4%, about 8. 0.5%, about 8. 0.6%, about 8.7%, about 8.8%, about 8.9%, about 9.0%, about 9.1%, about 9. 0.2%, about 9. 0.3%, about 9.4%, about 9.5%, about 9.6%, about 9.7%, about 9.8%, about 9.9% or about 10%, about 11%, about 12%, about 13%, about 14%, about 15%, about 16%, about 17%, about 18%, about 1%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%., about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

The selected penetration enhancer should be pharmacologically inert, non-toxic, and non-allergenic, have rapid and reversible onset of action, and be compatible with the compositions of the invention. Examples of penetration enhancers exemplified by transcutol P, ethyl alcohol, isopropyl alcohol, lauryl alcohol, salicylic acid, octolyphenylpolyethylene glycol, polyethylene glycol 400, propylene glycol, N-decylmethylsulfoxide, DMSO and azacyclo compounds.

In one exemplary embodiment, the present disclosure pertains to compositions for local administration of a compound of formula (I). As used herein, the term “local” refers to the limited area near the site of administration, generally the nerves at or near skin including the epidermis, the dermis, the dermatomes and the like, with no or limited systemic penetration beyond the skin.

Preferably, the topical delivery is designed to maximize drug delivery through the stratum corneum and into the epidermis or dermis or dermatome, and to minimize absorption into the circulatory system. More preferable are agents that may be used in topical formulations to prevent the passage of active ingredients or excipients into the lower skin layers. These so-called skin retardants have been readily developed for many over-the-counter (OTC) skin formulations, such as sunscreens and pesticides, where the site of action is restricted to the skin surface or upper skin layers. Research in the area of permeation enhancement or retardation is yielding valuable insights into the structure—activity relationships of enhancers as well as retardants (Asbill et al., 2000, Percutaneous penetration enhancers: local versus transdermal activity. Pharm. Sci. Tech. Today, 3(1):36-41; Kaushik, et al., 2008, Percutaneous permeation modifiers: enhancement versus retardation. Exp. Opin. Drug Del. 5(5):517-529; Trommer et al., 2006, Overcoming the Stratum Corneum: The Modulation of Skin Penetration. Skin Pharmacol. Physiol. 19:106-121) including such compounds as ketorolac stearate, Aminocaprolactam Analogues, Dicarboxylic acid ester, sodium citrate, and the like.

The compositions described herein can further comprise components usually admixed in such preparations. For example, the compositions may also include additional ingredients such as other carriers, moisturizers, oils, fats, waxes, surfactants, thickening agents, antioxidants, viscosity stabilizers, chelating agents, buffers, preservatives, perfumes, dyestuffs, lower alkanols, humectants, emollients, dispersants, sunscreens such as radiation blocking compounds or particularly UV-blockers, antibacterials, antifungals, disinfectants, vitamins, antibiotics, or other anti-acne agents, as well as other suitable materials that do not have a significant adverse effect on the activity of the topical composition. Additional ingredients for inclusion in the carrier are sodium acid phosphate moisturizer, witch hazel extract carrier, glycerin humectant, apricot kernel oil emollient, corn oil dispersant, and the like which are further detailed below. Those of skill in the art will readily recognize additional ingredients, which can be admixed in the compositions described herein.

According to another aspect, the pharmaceutical composition for topical administration or for transdermal application of a compound of formula (I) may comprise a thickening or gelling agent suitable for use in the compositions and methods described herein to increase the viscosity of the composition. Suitable agents (also known as gelling agents) are exemplified neutralized anionic polymers or neutralized carbomers, such as polyacrylic acid, carboxypolymethylene, carboxymethyl cellulose and the like, including derivatives of Ultrez 10, CARBOPOL® polymers, such as CARBOPOL® 940, CARBOPOL® 941, CARBOPOL® 954, CARBOPOL® 980, CARBOPOL® 981, CARBOPOL® ETD 2001, CARBOPOL® EZ-2 and CARBOPOL® EZ-3. (CARBOPOL is a registered trademark of Lubrizol Advanced Materials Inc., Cleveland, Ohio, USA). As used herein, a “neutralized carbomer” is a synthetic, high molecular weight polymer, composed primarily of a neutralized polyacrylic acid. Further, when a base is added to neutralize a carbomer solution, the viscosity of the solution increases. Also suitable are other known polymeric thickening agents, such as PEMULEN® polymeric emulsifiers, NOVEON® polycarbophils (PEMULEN and NOVEON are registered trademarks of Lubrizol Advanced Materials Inc.), and KLUCEL® (KLUCEL is a registered trademark of Hercules Inc., Wilmington, Del., USA). Additional thickening agents, enhancers and adjuvants may generally be found in Remington's The Science and Practice of Pharmacy as well as in the Handbook of Pharmaceutical Excipients (Arthur H. Kibbe ed. 2000). Alternatively, the pharmaceutical composition for topical administration or for transdermal application of a compound of formula (I) may comprise an anionic polymer thickening agent precursor, such as a carbomer, which has been combined with a neutralizer in an amount sufficient to form a gel or gel-like composition with a viscosity greater than 1000 cps as measured by a Brookfield RV DVII+ Viscometer with spindle CPE-52, torque greater than 10% and the temperature maintained at 25.degree. C. Alternatively, the anionic polymer thickening agent precursor may be combined with a neutralizer selected from the group consisting of: sodium hydroxide, ammonium hydroxide, potassium hydroxide, arginine, aminomethyl propanol, tetrahydroxypropyl ethylenediamine, triethanolamine (“TEA”), tromethamine, PEG-15 cocamine, diisopropanolamine, and triisopropanolamine, or combinations thereof in an amount sufficient to neutralize the anionic polymer thickening agent precursor to form a gel or gel-like composition in the course of forming the composition. The thickening agents or gelling agents are present in an amount sufficient to provide the desired rheological properties of the composition, which include having a sufficient viscosity for forming a gel or gel-like composition that can be applied to the skin of a mammal. The thickening agent or gelling agent is present in a total amount by weight of about 0.1%, about 0.25%, about 0.5%, about 0.75%, about 1%, about 1.25%, about 1.5%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, about 5.5%, about 5.75%, about 6.0%, about 6.25%, about 6.5%, about 6.75%, about 7.0%, about 7.25%, about 7.5%, about 7.75%, about 8.0%, about 8.25%, about 8.5%o, about 8.75%), about 9.0%, about 9.25%, about 9.5%, about 9.75%, about 10%, about 11%, about 11.5%, about 12%, about 12.5%, about 13%, about 13.5%, about 14%, about 14.5% or about 15%, and therebetween.

According to another aspect, the pharmaceutical composition for topical administration or for transdermal application of a compound of formula (I) may comprise an emollient. Suitable emollients are exemplified by mineral oil, mixtures of mineral oil and lanolin alcohols, cetyl alcohol, cetostearyl alcohol, petrolatum, petrolatum and lanolin alcohols, cetyl esters wax, cholesterol, glycerin, glyceryl monostearate, isopropyl myristate, isopropyl palmitate, lecithin, allyl caproate, althea officinalis extract, arachidyl alcohol, argobase EUC, butylene glycol, dicaprylate/dicaprate, acacia, allantoin, carrageenan, cetyl dimethicone, cyclome hicone, diethyl succinate, dihydroabietyl behenate, dioctyl adipate, ethyl laurate, ethyl palmitate, ethyl stearate, isoamyl laurate, octanoate, PEG-75, lanolin, sorbitan laurate, walnut oil, wheat germ oil, super refined almond, super refined sesame, super refined soyabean, octyl palmitate, caprylic/capric triglyceride and glyceryl cocoate. An emollient, if present, is present in the compositions described herein in an amount by weight of the composition of about 1% to about 30%, about 3% to about 25%, or about 5% to about 15%. Illustratively, one or more emollients are present in a total amount of about 1% by weight, about 2%, about 3%, about 4%, about 5%, about 6%, about 7%., about 8%, about 9%, about 10%, about 11%, about 12%, about 13%, about 14%, about 1%, about 16%, about 17%, about 18%, about 19%, about 20%, about 21%, about 22%, about 23%, about 24%, about 25%, about 26%, about 27%, about 28%, about 29%, or about 30%, and therebetween.

According to another aspect, the pharmaceutical composition for topical administration or for transdermal application of a compound of formula (I) may comprise an antioxidant. Suitable antioxidants are exemplified by citric acid, butylated hydroxytoluene (BHT), ascorbic acid, glutathione, retinol, a-tocopherol, .beta.-carotene, a-carotene, ubiquinone, butylated hydroxyanisole, ethyl enediaminetetraacetic acid, selenium, zinc, lignan, uric acid, lipoic acid, and N-acetylcysteine. An antioxidant, if present, is present in the compositions described herein in a total amount selected from the range of about 0.025% to about 1.0% by weight.

According to another aspect, the pharmaceutical composition for topical administration or for transdermal application of a compound of formula (I) may comprise an antimicrobial preservative. Illustrative anti-microbial preservatives include acids, including but not limited to, benzoic acid, phenolic acid, sorbic acids, alcohols, benzethonium chloride, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, propylparaben, sodium propionate or thimerosal. The anti-microbial preservative, if present, is present in an amount by weight of the composition of about 0.1% to about 5%, about 0.2% to about 3%, or about 0.3% to about 2%, for example about 0.2%, about 0.4%, about 0.6%, about 0.8%, about 1%, about 1.2%, about 1.4%, about 1.6%, about 1.8%, about 2%, about 2.2%, about 2.4%, about 2.6%, about 2.8%, about 3.0%, about 3.2%, about 3.4%, about 3.6%, about 3.8%, about 4%, about 4.2%, about 4.4%, about 4.6%, about 4.8%, or about 5%.

According to another aspect, the pharmaceutical composition for topical administration or for transdermal application of a compound of formula (I) may comprise one or more emulsifying agents. The term “emulsifying agent” refers to an agent capable of lowering surface tension between a non-polar and polar phase and includes self emulsifying agents. Suitable emulsifying agents can come from any class of pharmaceutically acceptable emulsifying agents exemplified by carbohydrates, proteins, high molecular weight alcohols, wetting agents, waxes and finely divided solids. The optional emulsifying agent, if present, is present in a composition in a total amount of about 1% to about 25%, about 1% to about 20%, or about 1% to about 15% by weight of the composition. Illustratively, one or more emulsifying agents are present in a total amount by weight of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%.

According to another aspect, the pharmaceutical composition for topical administration or for transdermal application of a compound of formula (I) may comprise a water-miscible solvent exemplified by propylene glycol. A suitable water-miscible solvent refers to any solvent that is acceptable for use in a pharmaceutical composition and is miscible with water. If present, the water-miscible solvent is present in a composition in a total amount of about 1% to about 95%, about 2% to about 75%, about 3% to about 50%, about 4% to about 40%, or about 5% to about 25% by weight of the composition.

According to another aspect, the pharmaceutical composition for topical administration or for transdermal application of a compound of formula (I) may comprise one or more alcohols. In a further embodiment, the alcohol is a lower alcohol. As used herein, the term “lower alcohol,” alone or in combination, means a straight-chain or branched-chain alcohol moiety containing one to about six carbon atoms. In one embodiment, the lower alcohol contains one to about four carbon atoms, and in another embodiment the lower alcohol contains two or three carbon atoms. Examples of such alcohol moieties include methanol, ethanol, ethanol USP (i.e., 95% v/v), n-propanol, isopropanol, n-butanol, isobutanol, sec-butanol, and tert-butanol.

Ethanol may be used as dehydrated alcohol USP, alcohol USP or in any common form including in combination with various amounts of water. If present, the alcohol is present in an amount sufficient to form a composition which is suitable for contact with a mammal. For example, in a total amount by weight of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%.

Another embodiment pertains to pharmaceutical compositions comprising a compound of formula (I) formulated for parenteral administration by injection. The injectable pharmaceutical compositions of the present disclosure comprise a suitable carrier solution exemplified by sterile water, saline, and buffered solutions at physiological pH. Suitable buffered solutions are exemplified by Ringer's dextrose solution and Ringer's lactated solutions. The carrier solution may comprise in a total amount by weight of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%>, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%>, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%), about 9.3%, about 9.4%, about 9.5%, about 9.6%, about 9.7%, about 9.8%, about 9.9% or about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63% , about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

According to one aspect, the injectable pharmaceutical compositions may additionally incorporate one or more non-aqueous solvents exemplified by propylene glycol, polyethylene glycol, vegetable oils such as olive oil, and injectable organic esters exemplified by ethyl oleate.

According to another aspect, the injectable pharmaceutical compositions may additionally incorporate one or more of antimicrobials, anti-oxidants, chelating agents and the like.

The injectable pharmaceutical compositions may be presented in unit-dose or multi-dose containers exemplified by sealed ampules and vials. The injectable pharmaceutical compositions may be stored in a freeze-dried (lyophilized) condition requiring the addition of a sterile liquid carrier, e.g., sterile saline solution for injections, immediately prior to use.

Another embodiment pertains to pharmaceutical compositions comprising a compound of formula (I) formulated for oral administration. The oral pharmaceutical compositions may be provided as capsules or tablets; as powders or granules; as solutions, syrups or suspensions (in aqueous or non-aqueous liquids). Tablets or hard gelatine capsules may comprise, for example, lactose, starch or derivatives thereof, magnesium stearate, sodium saccharine, cellulose, magnesium carbonate, stearic acid or salts thereof. Soft gelatine capsules may comprise, for example, vegetable oils, waxes, fats, semisolid, or liquid polyols, etc. Solutions and syrups may comprise, for example, water, polyols and sugars. The compound of formula (I) may be coated with or admixed with a material (e.g., glyceryl monostearate or glyceryl distearate) that delays disintegration or affects absorption of the active agent in the gastrointestinal tract. Thus, for example, the sustained release of an active agent may be achieved over many hours and, if necessary, the active agent can be protected from being degraded within the gastrointestinal tract. Taking advantage of the various pH and enzymatic conditions along the gastrointestinal tract, pharmaceutical compositions for oral administration may be formulated to facilitate release of an active agent at a particular gastrointestinal location.

### EXAMPLES

### Example 1

### Preparation of 4-(1,1,1,3,3,3-hexafluoro-2-hydroxypropan-2-yl)-N,N-dipropylbenzenesulfonamide (compound 1)

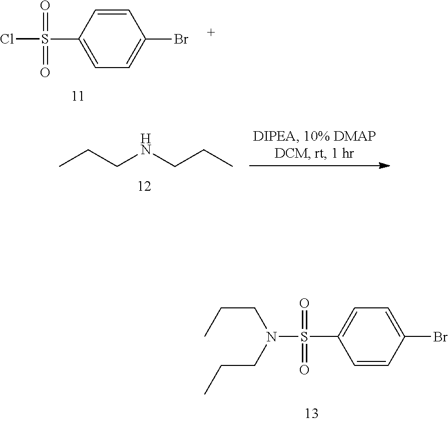 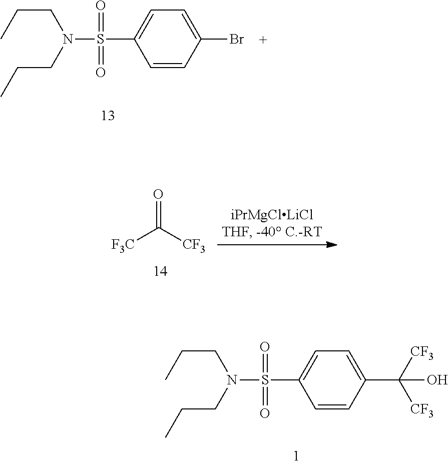 [SMILES: CCCN(CCC)S(=O)(=O)c1ccc(Br)cc1 ; O=C(C(F)(F)F)C(F)(F)F ; CCCN(CCC)S(=O)(=O)c1ccc(C(O)(C(F)(F)F)C(F)(F)F)cc1] [0120] a) A mixture of 4-bromobenzenesulfonyl chloride (compound 11) (11.98 g, 46.9 mmol) and DMAP (0.57 g, 4.7 mmol) were purged with nitrogen and dissolved in 100 mL DCM, and dipropylamine 12 (7.25 mL, 52.9 mmol) was added slowly followed by DIPEA (41 mL, 235 mmol) and the reaction mixture was stirred at room temperature overnight to form 4-bromo-N,N-dipropylbenzenesulfonamide (compound 13). After 19 hours the solution was concentrated in vacuo and the resulting powder was purified by silica gel flash chromatography (15% EtOAc in hexanes) to afford 14.8 g of compound 13 as a white crystalline powder (46.2 mmol, 98% yield). 1H NMR (400 MHz, Chloroform-d) δ 7.75-7.60 (m, 4H), 3.17-3.02 (m, 4H), 1.67-1.47 (m, 4H), 0.88 (t, J =7.4 Hz, 6H). 13C NMR (101 MHz, CDCl₃) δ 139.4, 132.2, 128.6, 127.1, 50.0, 22.0, 11.2. HRMS (EI) m/z calc. for [C₁₂ H 18 BrNO 2 S]+=319.0242, found 319.0246.
b) Compound 13 (101 mg, 0.32 mmol) was dissolved in 2 mL of distilled THF and cooled to −78° C. Hexafluoroacetone sesquihydrate (0.3 mL, 2.6 mmol) was dropped onto concentrated sulfuric acid and the liberated 1,1,1,3,3,3-hexafluoropropan-2-one (compound 14) was trapped in THF at −78° C. 0.35 mL (0.43 mmol) of iPrMgCl.LiCl (Turbo Grignard) was added dropwise and the solution was stirred for 20 minutes before being warmed to room temperature. The solution was stirred for 2.5 hours and then the solution of 1,1,1,3,3,3-hexafluoropropan-2-one (compound 14) was added dropwise. The reaction was stirred for 2 hours before being quenched with saturated NH₄ Cl. The organic layer was concentrated in vacuo and the crude mixture was purified by silica gel flash chromatography (10% EtOAc in hexanes) to give 23 mg of compound 1 (0.056 mmol, 18% yield). 1H NMR (400 MHz, Chloroform-d) δ 7.89 (s, 4H), 3.17-3.08 (m, 4H), 1.66-1.52 (m, 4H), 0.89 (t, J=7.4 Hz, 6H). 13C NMR (101 MHz, CDCl3) δ 142.22, 133.47, 127.52, 127.13, 123.77, 120.91, 50.05, 22.04, 11.07. 19F NMR (376 MHz, CDCl3) δ −75.37. HRMS (ESI) calc. for [C₁₅H₁₉F₆NO₃S⁺Na⁺]⁺430.0882, found 430.0892.

### Example 2

### Preparation of 4-(1,1,1,3,3,3-hexafluoro-2-hydroxypropan-2-yl)-N,N-dimethylbenzenesulfonamide (compound 2)

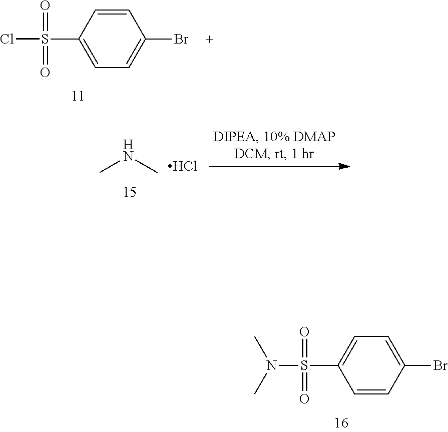 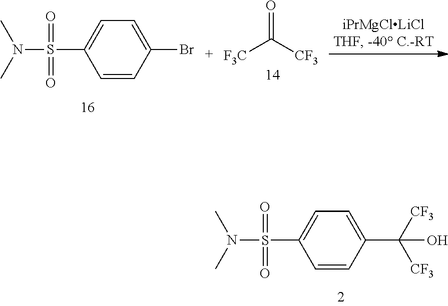 [SMILES: CN(C)S(=O)(=O)c1ccc(Br)cc1 ; O=C(C(F)(F)F)C(F)(F)F ; CN(C)S(=O)(=O)c1ccc(C(O)(C(F)(F)F)C(F)(F)F)cc1] [0123] a) A mixture of 4-Bromobenzenesulfonyl chloride (2.203 g, 8.6 mmol) (compound 11), DMAP (0.116 g, 0.95 mmol), and N,N-dimethylamine hydrochloride (0.743 g, 9.1 mmol) were purged with nitrogen. 25 mL of DCM was added, and the solution was stirred until all solids dissolved. DIPEA (9.1 mL, 52 mmol) was added slowly and the reaction was stirred at room temperature for 5 hours. The resulting solution was concentrated in vacuo and the residue was purified by silica gel flash chromatography (20% EtOAc in hexanes) to afford 2.17 g of product (8.2 mmol, 95% yield) as a white powder. 1H NMR (600 MHz, Chloroform-d) δ 7.73-7.68 (m, 2H), 7.68-7.62 (m, 2H), 2.72 (s, 6H). 13C NMR (151 MHz, CDCl₃) δ 134.71, 132.30, 129.17, 127.71, 37.83. HRMS (El) m/z calc. for [C₈H₁₀BrNO₂S]⁺=262.9616, found 262.9623.
b) Compound 16 (199 mg (0.753 mmol) was dissolved in 2 mL of distilled THF and cooled to −78° C. nBuLi (0.52 mL, 0.749 mmol) was then added dropwise and the solution was allowed to stir for 10 minutes at −78° C. before the solution of 1,1,1,3,3,3-hexafluoropropan-2-one 14 in THF was added dropwise. The reaction was then allowed to warm to room temperature and stirred for 2 hours. The reaction was quenched with saturated NH4Cl, diluted with water and extracted with EtOAc. The combined extracts were washed with water and then dried over MgSO4 and filtered. The solvent was removed in vacuo and compound 2 was isolated by silica gel flash chromatography (1% MeOH in DCM) to yield 192 mg of the title compound (0.546 mmol, 73% yield). 1H NMR (400 MHz, Chloroform-d) δ 7.97-7.84 (m, 4H), 2.77 (s, 6H). 13C NMR (101 MHz, CDCl3) δ 137.97, 133.85, 127.80, 127.57, 123.75, 120.88, 37.83. 19F NMR (376 MHz, CDCl3) δ −75.34. HRMS (El) calc. for [C₁₁H₁₁F₆NO₃S]+=351.0364, found 351.0352.

### Example 3

### Preparation of 4-(1H-indazol-5-yl)-N,N-dipropylbenzenesulfonamide (compound 3)

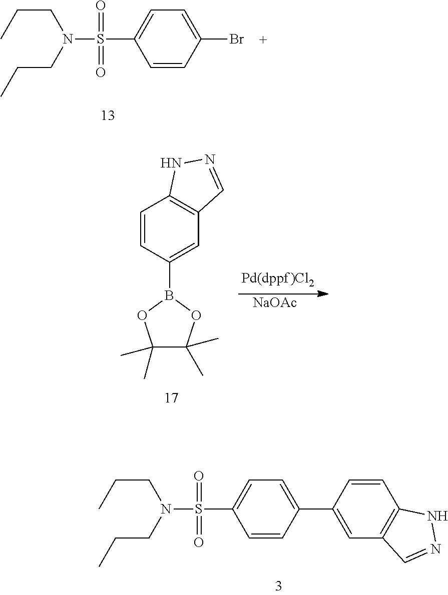 [SMILES: CCCN(CCC)S(=O)(=O)c1ccc(Br)cc1 ; CC1(C)OB(c2ccc3[nH]ncc3c2)OC1(C)C ; CCCN(CCC)S(=O)(=O)c1ccc(-c2ccc3[nH]ncc3c2)cc1 ; "NaOAc"]

Compound 13 (362 mg, 1.13 mmol) and 5-(4,4,5,5-tetramethyl-1,3,2-dioxaborolan-2-yl)-1H-indazole (compound 17) (320 mg, 1.31 mmol), Pd(dppf)Cl₂ (90 mg, 0.11 mmol), and NaOAc (293 mg, 3.371 mmol) were added and purged with nitrogen. The solids were then dissolved in 10 mL of MeCN and 1.0 mL of de-ionized water was added. The resulting red-brown solution was stirred at 100° C. for 16 hours. The resulting solution was diluted with water and EtOAc and separated, and the aqueous layer was extracted with EtOAc and the combined organic extracts were concentrated in vacuo. The crude residue was purified twice by silica gel flash chromatography (30-45% EtOAc in hexanes with 1% TEA, then 1-4% MeOH in DCM) after which the product was subjected to reverse phase column chromatography (5-95% MeCN in water), yielding 100 mg of the title compound (280 mmol, 25% yield). ¹H NMR (400 MHz, Chloroform-d) δ 8.19 (s, 1H), 8.02 (dd, J=1.7, 0.9 Hz, 1H), 7.94-7.87 (m, 2H), 7.80-7.73 (m, 2H), 7.73-7.60 (m, 2H), 3.20-3.11 (m, 4H), 2.20 (s, 1H), 1.69-1.55 (m, 4H), 0.92 (t, J=7.4 Hz, 6H). ¹³C NMR (101 MHz, CDCl₃) δ 145.33, 139.85, 138.43, 135.55, 132.84, 127.69, 127.65, 126.72, 123.94, 119.64, 110.36, 50.14, 22.12, 11.23. HRMS (EI) calc. for [C₁₉H₂₃N₃O₂S]+=357.1511, found 357.1510.

### Examples 4-6

Compounds 4, 5 and 6 were also prepared by reacting compound 13 and the respective boronic esters (18, 19 and 20, respectively) under Suzuki cross coupling reaction conditions, via the procedure as described in Example 3.

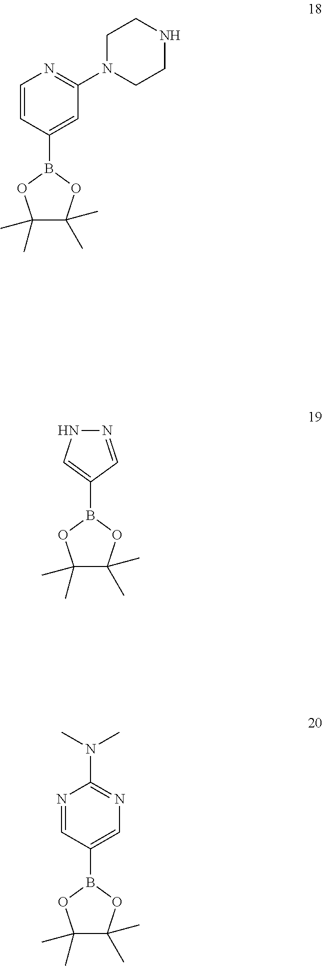

### Example 7

The following methods and materials were used to test the activity of Compound 1.

### YO-PRO-1 Dye Uptake

After plating into 96 well plates, cell culture media was removed and BV-2 cells were incubated in extracellular solution (ECS) (140 mM NaCl, 4.5 mM KCl, 1.3 mM CaCl2, 10 mM HEPES, and 33 mM glucose; pH7.35, osmolarity 315 mOsm) containing YO-PRO-1 dye (2.5 μM, Invitrogen). After a 5 minute baseline recording, cells were stimulated with 3′-O-(4-benzoyl)benzoyl adenosine 5′-triphosphate (BzATP) (150 μM, Toronto Research Chemicals) and dye-uptake was recorded for 30 minutes. Cell viability was assessed immediately after the 30 minute recording by application of ionomycin (1 μM, Sigma). Cells that did not respond to ionomycin were excluded from the analysis. YO-PRO-1 fluorescent emission (491/509) was detected using a FilterMax F5 plate reader (Molecular Devices). Drugs used include (−)-norepinephrine (100 pM-1 μM, Sigma), probenecid (1 mM, Life Technologies), and Compound 1 (10 nM, Derksen et al). Drugs were bath applied in ECS containing YO-PRO-1 dye and incubated at 37° C. for 10 minutes prior to BzATP stimulation. Fluorescent emission at 30 minutes post-BzATP was calculated as an average of the entire imaging field as percent change from baseline, and responses at 30 minutes were averaged over multiple independent experiments. Individual traces of BV-2 YO-PRO-1 dye uptake were taken at 10 minute intervals and represent the average response of all BV-2 cells in the recorded view from a subset of experiments.

### BV-2 Microglia-Like Cell Culture

BV-2 microglia-like cells were maintained in DMEM (Gibco) containing 5% fetal bovine serum (Gibco) and 1% penicillin-streptomycin (Gibco) with 5% CO2. BV-2 microglia-like cells were found to be free of mycoplasma contamination using a MycoFluor™ Mycoplasma Detection Kit (Molecular Probes).

### Opioid Withdrawal Experiments:

### Animals

Adult male C57BL/6J (7-9 weeks) mice were used. Mice were housed under 12/12 hour light/dark cycles with ad libitum access to food and water. Mice were randomly allocated to different test groups. All experiments were approved by the University of Calgary Animal Care Committee and are in accordance with the guidelines of the Canadian Council on Animal Care.

### Behavioral Assessment of Morphine Withdrawal

Morphine sulfate (MS, PCCA) prepared in 0.9% sterile saline solution was injected i.p. into male C57BLJ6J (20-30g) mice. Mice received ascending doses of morphine (i.p.) at 8 hours intervals: day 1, 10mg/kg; day 2, 20 mg/kg; day 3, 30 mg/kg; day 4, 40mg/kg. On day 5, mice received a morning injection of morphine (50 mg/kg). A subset of mice received probenecid (15, 25, and 50 mg/kg i.p., Invitrogen) or Compound 1 (0.1, 0.5 and 1 mg/kg) 1 hour following morphine. Mice then received i.p. naloxone (2 mg/kg, naloxone hydrochloride dihydrate, Sigma) 2 hours following morphine to rapidly induce opioid withdrawal. Control mice received equivalent volumes of 0.9% saline or appropriate vehicle solution (0.1% DMSO in saline) and were challenged with naloxone on day 5. Withdrawal behaviors were then scored for 30 minutes post naloxone injection. Behaviors were scored for occurrence and then scaled for severity at 5-minute intervals. Behaviors that were assigned a standardized score between 0-3 depending on severity were jumping, headshakes, wet-dog shakes, chewing/licking, and teeth chattering. Behaviors that were scored 1 point for presence during a 5-minute interval include piloerection and tremors/twitching. The mice were then weighed following the 30 minutes, and the weight difference was assigned a standardized score dependent on the weight change between 0-3 as well. The scaled and present/absent signs were compiled over the 30 minutes to produce a composite withdrawal score.

### Statistics

All data are presented as the mean±standard error mean (s.e.m), and each circle represents an individual animal or experiment. Tests of statistical difference were performed with GraphPad Prism 6 using either an ordinary one-way ANOVA with posthoc Dunnet test, or a two-way ANOVA with post hoc Sidak test. Sample sizes are consistent with those reported in similar studies and provide sufficient power to detect changes with the appropriate statistical analysis. For all experiments, a criterion a level was set at 0.05.

### Opioid Seeking and Relapse Experiments:

### Animals

Adult (8 to 10 weeks old at the beginning of the experiments) wild-type male and female Long Evans rats (250-350g) were used. Animals were group housed prior to jugular vein catheter implant surgery with two to three animals per cage on a 12/12 hours dark/light cycle (lights on at 7:00 AM). Rats were acclimated to the animal facility holding rooms for at least 7 days before any manipulation. After catheter implantation, animals were single housed to prevent damage to the harnesses. All experiments were performed during the light cycle. Rats received food and water ad libitum until 2 days prior to starting the behavioral studies, when food restriction (16 g of rat chow per day) started and continued until the end of the experiments.

All procedures were approved by Washington University Committee in accordance with the National Institutes of Health Guidelines for the Care and Use of Laboratory Animals.

### Surgeries

All surgeries were performed under isoflurane (2.5/3 MAC) anesthesia using sterile aseptic techniques. For intravenous (IV) self-administration (SA), animals were implanted with sterile catheters in the right jugular vein and connected to a harness placed over the torso. The harness allowed for minimal stress while connecting and disconnecting the animal from infusion lines. The catheter was kept patent by daily infusions of gentamicin mixture (1.33 mg/ml).

To minimize post-surgical pain, animals received a daily subcutaneous (s.c.) injection of 8 mg/kg enrofloxacin and 5 mg/kg carprofen for 2 consecutive days together with carprofen chewable tablets. Behavioral experiments started 1 week after the catheter implantation.

### Operant Intravenous Self-Administration

Equipment: Rat self-administration was conducted using operant-conditioning chambers (Med Associates) equipped with two retractable levers with a food magazine connected to a food pellet dispenser between them. Two cue lights were positioned above the levers, and one house light was positioned on the top left-hand wall. During self-administration sessions both levers (correct and incorrect lever) were extended out with white cue light turned on only above the correct lever. Presses on the correct lever resulted in reward delivery and a 20 s time-out period during which correct and incorrect lever were retracted, and cue light was turned off. Presses on the incorrect lever did not result in any changes in the environment.

Behavioral procedure: Animals were placed in operant boxes and exposed to fixed ratio (FR) 1 schedule of reinforcement (1lever press results in the delivery of one food pellet) for 2 hours daily (or until the rat obtained a maximum of 60 rewards during the session) for at least 5 sessions.

After acquisition of the task, rats received a jugular catheter implantation (see procedure above) and recovered for a week, to avoid post-surgical pain and distress. Animals were then placed in operant boxes and their harnesses were gently tethered to a drug infusion line connected to an infusion pump. Animals were exposed to a daily 2-hour intravenous self-administration session, during which a press on the active lever resulted in an intravenous 1.5 mg/kg/infusion of morphine. Rats underwent 5 sessions of FR1 schedule of reinforcement, followed by 3 sessions of FR2 (2 lever presses result in reward delivery), and then 3 sessions of FR5 (5 lever presses result in reward deliver). After the last FR5 session, animals were exposed to extinction sessions (1hour session daily for 10 days) during which the animals remained untethered, both levers were extended, and both the light cues above levers and the infusion pump (sound) were turned off. Presses on both “active” lever and “inactive” lever had no consequence.

To assess cue-induced reinstatement, animals were placed in the operant boxes after extinction sessions, tethered to the infusion lines, and both the cue-lights associated with drug availability and the sound of the infusion pump were turned back on. During the 1-hour reinstatement session none of the presses on both correct and incorrect levers resulted in drug infusion. Compound 1 (0.5 mg/kg diluted in 0.1% DMSO) was injected intraperitoneally (i.p.) in three different treatment paradigms: 1) drugs were given 1 hour prior to each extinction session but not on the day of reinstatement, 2) 1 hr before reinstatement test, or 3) 1 hour prior to each extinction session and before reinstatement.

### Analysis

All experiments were performed at least twice, including each treatment condition to prevent an unspecific day/condition effect. Treatment groups were randomly assigned to animals prior to testing. All data are expressed as mean±SEM. Sample sizes (N number) always refers to value obtained from an individual animal. Treatment groups were randomly assigned to animals prior to testing. After assessing the normality of sample data using Shapiro-Wilk test, statistical significance was determined by two-way repeated-measures ANOVA followed by two-tailed Sidak post hoc test. Data was analyzed using GraphPad Prism 8.1.0.

Conditioned place aversion (CPA) Experiments:

### Animals

Adult male C57BL/6 mice (7-14 weeks old) were used for conditioned place aversion experiments (CPA). Animals were group housed under reverse light-dark cycle with ad libitum access to food and water and were randomly allocated to different test groups. All procedures were approved by the University of Calgary and Washington University Animal Care Committees and are in accordance with the guidelines of the Canadian Council on Animal Care and the US National Institutes of Health Guide for the Care of Use of Laboratory Animals.

### Conditioned Place Aversion (CPA)

Place conditioning was performed in a custom-built clear plexiglass apparatus (28×28×19 cm) divided into two conditioning chambers distinct in tactile and visual cues. All locomotor behaviours were recorded using monochrome GigE cameras and quantified offline using EthoVision XT 11 software (Noldus). The CPA score was calculated by subtracting time spent in the conditioned chamber during baseline (pre-test) from time spent in the same chamber postconditioning. Mice were placed in a two-chamber conditioning apparatus and allowed free access to both chambers for 15 min to assess baseline preference. Mice were treated with escalating doses of morphine, control animals received equivalent volumes of 0.9% sterile saline. Two hours after the final morphine or saline injection on day 5, mice were injected with naloxone and confined to one side of the conditioning apparatus for 30 min. The conditioned place aversion (CPA) test occurred 1 and 7 days post-conditioning, where mice were allowed free access to the conditioning apparatus for 15 min. Mice were injected with Compound 1 (0.5 mg per kg, i.p.) or vehicle control 1 h before conditioning.

### Data Analysis

Unpaired t tests were applied to compare differences between two groups, one-way ANOVA with Bonferroni post hoc tests were applied to compare differences between three or more groups. Treatment groups were randomly assigned to animals prior to testing. All data are expressed as mean±SEM. Sample sizes (N number) refers to value obtained from an individual animal. Treatment groups were randomly assigned to animals prior to testing. After assessing the normality of sample data using Shapiro-Wilk test, statistical significance was determined by two-way repeated-measures ANOVA followed by two-tailed Sidak post hoc test. All data were presented as mean±SEM and analyzed using GraphPad Prism 9 (GraphPad Software, Inc., California).

### Results

Compound 1, 2 , 3, 4, 5 and 6 blocks Panx1 channel function

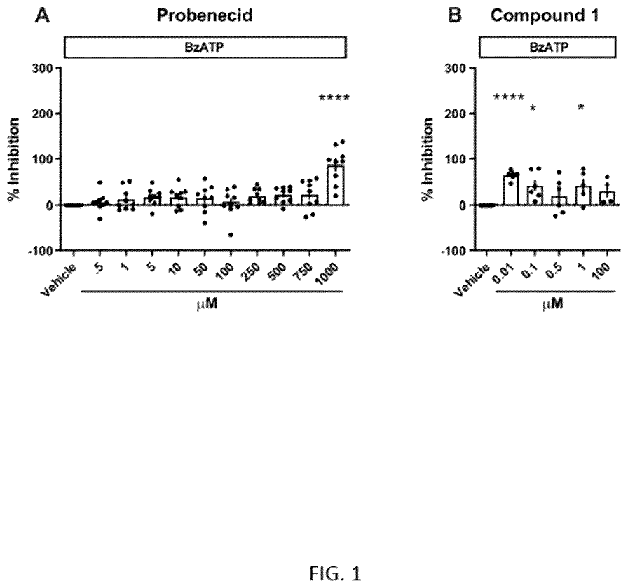

The compounds tested were designed to more readily penetrate the blood brain barrier and were therefore predicted to require a lower dose to produce similar effects. Prior to testing the compounds in animals, the compounds were first tested in BV2 microglia-like cells using the YO-PRO-1 Dye Uptake Assay. By using this assay, Panx1 channel activity in response to BzATP stimulation of P2X7, an upstream receptor that causes Panx1 pore formation can be assessed. YO-PRO-1 is a large molecular weight dye that can only enter the cell through large pore ion channels such as Panx1, therefore its uptake correlates with Panx1 channel opening. BzATP stimulation causes a significant increase in YO-PRO-1 dye uptake in cells pre-treated with ECS and vehicle solution (0.1% DMSO) (FIG. 1, B).

Pre-treatment with Compound 1 significantly blocked Panx1 channel activity at a concentration 100,000-fold lower than probenecid (FIG. 1). This finding indicates that Compound 1 is more potent than probenecid at inhibiting Panx1 channel opening in BV-2 cells.

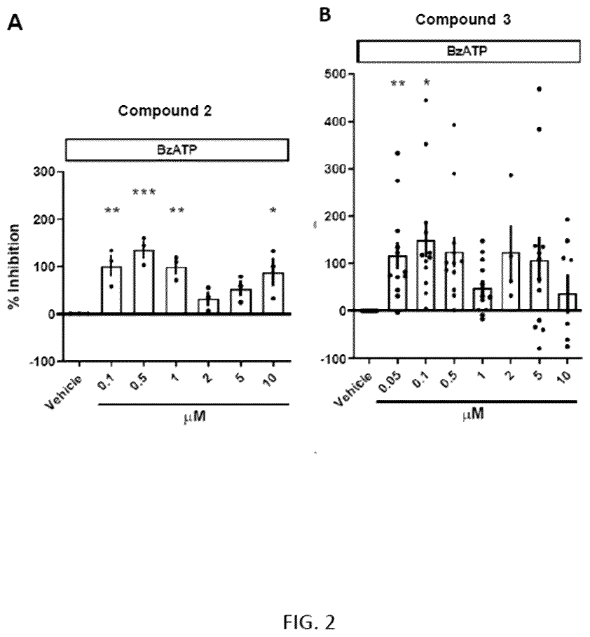

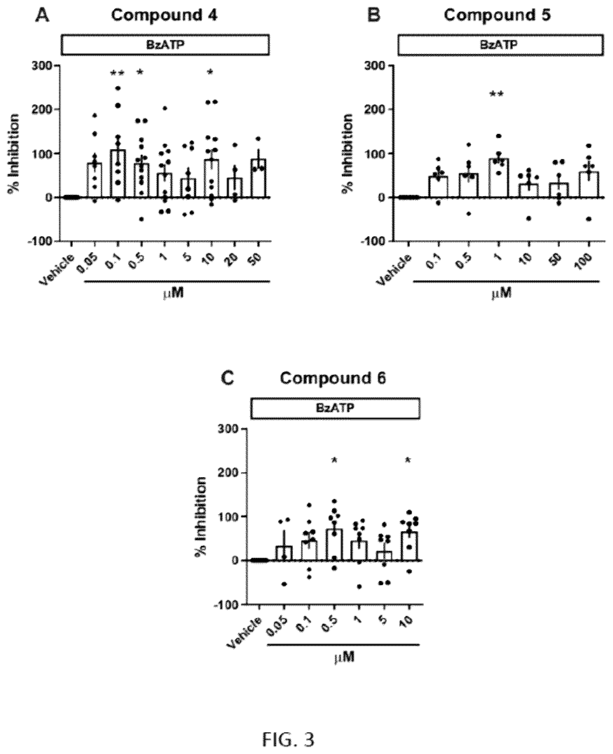

Pre-treatment with Compounds 2 and 3 (FIG. 2) and Compounds 4, 5, and 6 (FIG. 3) also significantly blocked Panx1 channel activity.

### Compounds 1 and 5 Inhibit Panx1 Channel Activity.

To further compare the effects of probenecid, Compounds 1 and 5 on Pannexin-1 channels Human Panx-1 was expressed in embryonic kidney-293T cells (293T) and channel activity was assessed at various doses of each compound.

### Molecular Biology

Rattus norvegicus Pannexin-1 (rPanx1) complementary DNA (cDNA) was cloned between BamHI and SalI sites in a pRK5 expression vector (pRK5-rPanx1). Enhanced green fluorescent protein (EGFP) plasmid (pEGFP-C1) was commercially purchased (addgene).

### Cell Culture & Transfection

Human embryonic kidney-293T cells (293T) were purchased from ATCC and routinely maintained in the laboratory. Adherent 293T cells were grown in Dulbecco's modified Eagle's medium (Thermo Fisher) supplemented with 10% fetal bovine serum (Thermo Fisher) and 1% penicillin-streptomycin (10,000 U/mL; Thermo Fisher) at 37° C. in a 5% CO₂ humidified growth incubator. 293T cells between the passages 6-20 were used for experiments. For patch-clamp recordings, 293T cells reaching 40-60% confluency on 35 mm culture plates were transiently transfected using Lipofectamine 2000 (Thermo Fisher) 36-48 hours before experiments, using the manufacturer's protocol. 293T cells were transfected with 2.5 μg pRK5-rPanx1 and 0.5 μg pEGFP-C1 cDNAs at a mixed ratio of 5:1 to identify positively transfected cells.

### Patch-clamp Electrophysiology

On the day of experiments, transiently transfected 293T cells overexpressing rPanx1 and EGFP cDNAs were seeded onto poly-d-lysine coated glass coverslips at least 2 hours before recordings. 293T cells were maintained at 30° C. throughout the duration of each recording and voltage-clamped in whole-cell configuration using a Multiclamp 700B amplifier (Axon Instruments). Data was acquired using Clampex (v.10) software and an Axon Digidata 1550A digitizer (Axon Instruments) at 10 kHz, with currents analyzed offline in Clampfit software (v.10.7). Patch pipettes were pulled from 1.5/0.86 mm [outer diameter/inner diameter] borosilicate glass (Sutter Instrument) using a P-1000 Micropipette Puller (Sutter Instrument) and had resistances of 3-5 MΩ. Patch pipettes contained (in mM) 4 NaCl, 1 MgCl₂, 0.5 CaCl₂, 30 TEA-Cl, 100 CsMeSO₄ 10 EGTA, 10 HEPES, 3 ATP-Mg²⁺, and 0.3 GTP-Tris, pH=7.3 (with KOH), ˜290 mosmol/liter (solutes purchased from Sigma). Recordings were performed on a Zeiss Axio Observer Z1 inverted epifluorescence microscope using a 40×/0.6 air-immersion objective (Zeiss), 470 nm light-emitting diode (LED) (Zeiss) and a 38 HE filter set (Zeiss) to visualize EGFP signal. 293T cells were under continuous bath perfusion with extracellular solution containing (in mM) 140 NaCl, 3 KCl, 2 MgCl₂, 2 CaCl₂, 10 D-(+)-Glucose, and 10 HEPES, pH=7.3 (with NaOH), ˜305 mosmol/liter (solutes purchased from Sigma).

Drug concentrations for patch-clamp experiments were selected from results obtained in the dye-uptake assay. Compounds were dissolved in DMSO (Sigma) to make a 10mM stock solution and diluted to final concentrations in extracellular solution. Final concentrations of DMSO in extracellular solutions were from 0.001 to 0.1% v/v, with highest percentage of DMSO being used for vehicle control recordings.

293T cells were voltage clamped at −60 mV in whole-cell configuration and exposed to a 300 ms voltage ramp (−80 to +80 mV) before stepping back to −60 mV to record rPanx1 voltage-sensitive currents. Baseline currents were first measured in extracellular solution before perfusion of drug compounds in incrementally increasing concentrations. If access resistance eclipsed ≥25 MΩ, cells were discarded from analysis. Current-voltage (IV) relationship plots incorporate averaged traces of pooled cell recordings. Statistical analysis of current amplitude, and percent of baseline plots were performed with one-way repeated measures ANOVA (Graphpad Prism 8). P-values of <0.05 were deemed significant. Note that the full inhibition of transfected current by maximum doses of Probenecid represents 100% block of Panx1. Remaining current (expressed as % of baseline) is comprised of other ion channels in the 293T cells that are insensitive to the Panx1 blockers.

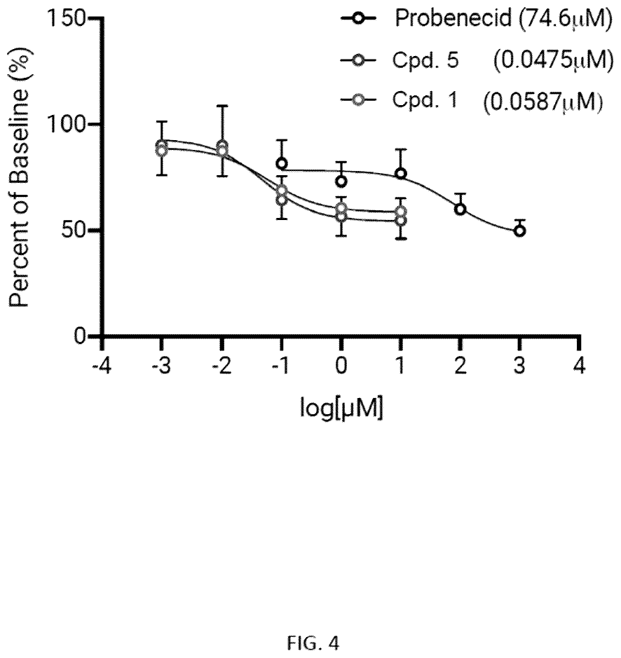

Compounds 1 and 5 are more potent Panx1 than Probenecid (FIG. 4).

### Compound 1 Attenuates Naloxone-Precipitated Withdrawal Behaviors in Mice

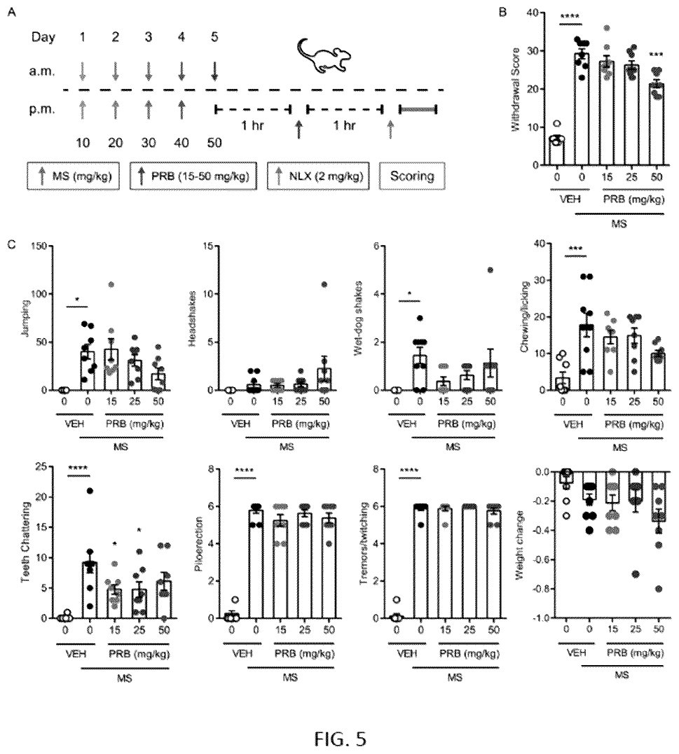

To compare the effects of probenecid and Compound 1 on withdrawal behaviors in mice, a naloxone-precipitated model of morphine withdrawal was used (FIG. 5A). In this model, naloxone injection in mice treated with ascending doses of morphine produces robust withdrawal behaviors such as jumping, headshakes, wet-dog shakes, chewing/licking, teeth chattering, piloerection and tremors/twitching (FIG. 5 B, C). Mice that were treated with 50 mg/kg, but not 15 or 25 mg/kg of probenecid 60 minutes prior to naloxone-precipitated withdrawal showed a significantly reduced withdrawal score (FIG. 5 B), demonstrating that probenecid is effective at attenuating withdrawal behaviors in mice.

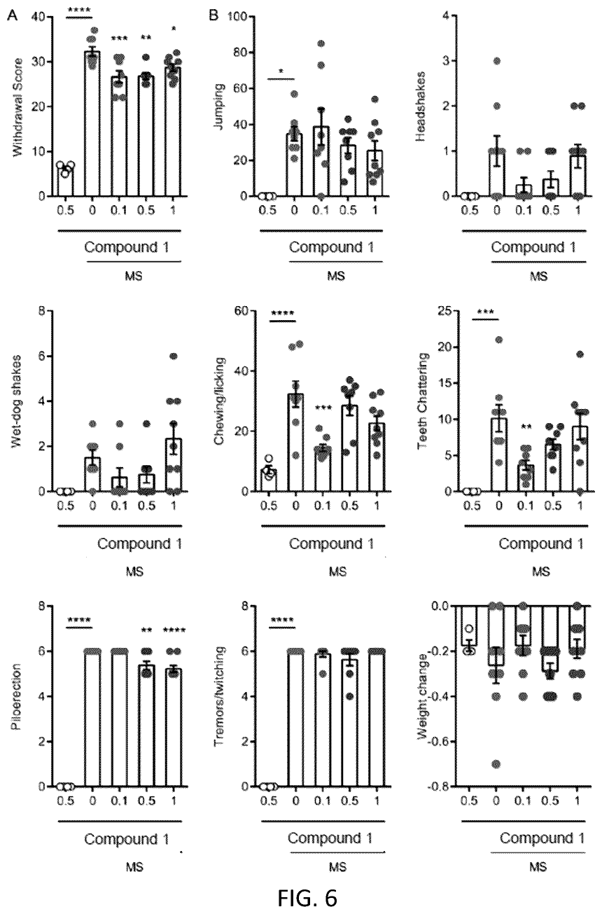

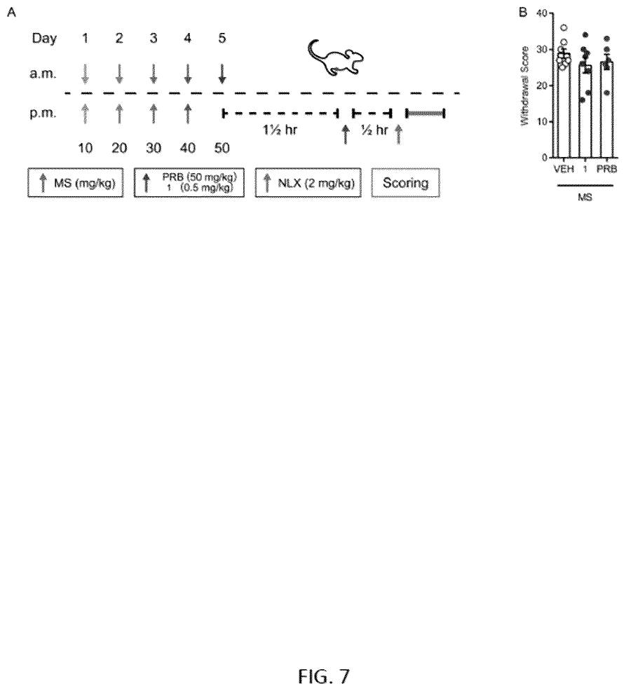

Pre-treatment with Compound 1 60 minutes prior to naloxone injection significantly attenuated withdrawal behaviors at 0.1, 0.5 and 1 mg/kg, doses 50-500 fold lower than probenecid (FIG. 6 A, B). These data demonstrate that the structural modifications made to probenecid allow for efficacious withdrawal attenuation at a significantly lower dose. To assess whether the pharmacokinetics of Compound 1 differed from probenecid, naloxone-precipitated morphine withdrawal model was repeated, but instead administered probenecid (50 mg/kg) and Compound 1 (0.5 mg/kg) 30 minutes prior to withdrawal (FIG. 7A). Both probenecid and Compound 1 were not effective at attenuating withdrawal behaviors when given 30 minutes before withdrawal (FIG. 7B). Therefore, Compound 1 attenuates withdrawal behaviors in mice with a similar temporal onset as probenecid, but at concentrations 50-500 fold lower.

### Compound 1 Reduces Motivational Opioid Seeking Behaviours

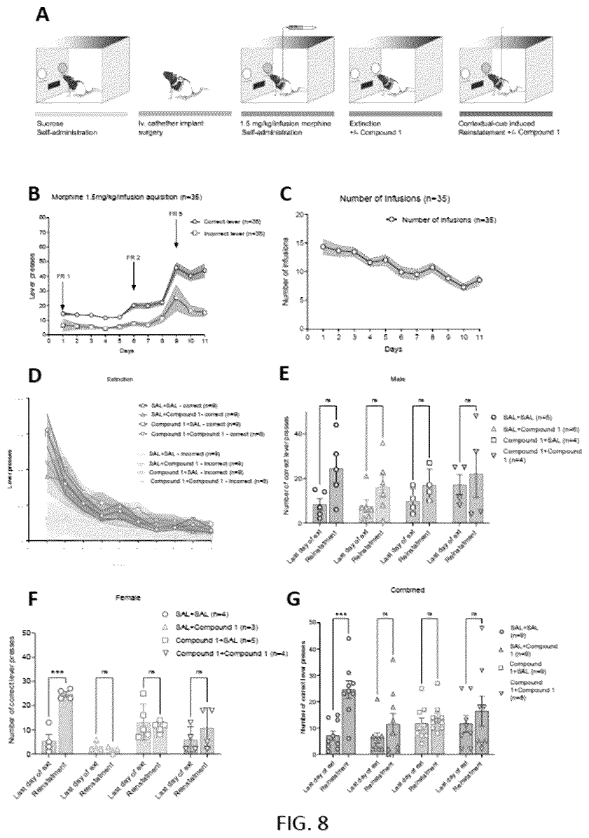

In adult male and female Long Evans rats, the effect of Compound 1 on cue-induced opioid reinstatement, a model of opioid seeking and relapse, was examined (FIG. 8A). For this behavioural test, a fixed ratio (FR) schedule FR1, FR2 and FRS, where 1, 2, 5 lever presses, respectively, in response to a light cue resulted in morphine infusions (FIG. 8B-C) was used.

The number of correct lever presses needed to obtain morphine as a reward exponentially increased (FIG. 8B), indicating that rats readily acquired motivation to seek opioids. Rats were then divided into two groups, where one group received Compound 1 throughout the extinction period, when the light cue was off, and no morphine was given. The second group received an additional dose of Compound 1 before the reinstatement phase, when light cue-paired lever presses led to morphine infusions. Rats administered Compound 1 did not show a reduction in time required for extinction of morphine seeking behaviour (FIG. 8D). During the reinstatement phase, rats treated with Compound 1 throughout extinction, or extinction and prior to reinstatement, displayed a significant reduction in the number of lever presses as compared with morphine dependent rats treated with saline control (FIG. 8G). This effect is conserved across the sexes, where Compound 1 reduced male (FIG. 8E) and female rats (FIG. 8F) decreased the number of lever presses for morphine infusions during reinstatement.

### Compound 1 Reduces Opioid Withdrawal Aversion

Opioid withdrawal is aversive and manifests as physical signs and behaviours. To assess aversion, conditioned place avoidance (CPA) was used. CPA is a well-established behavioral test in which rodents avoid or spend less time in a chamber they previously experienced naloxone precipitated opioid withdrawal.

To examine withdrawal aversion, the CPA test was used wherein morphine-dependent mice underwent naloxone-precipitated withdrawal while confined to one side of a two-chamber place conditioning apparatus; these mice displayed robust withdrawal behaviours during the 30 min conditioning session. When allowed free access to both chambers 1 day after naloxone-precipitated withdrawal, morphine-dependent mice administered vehicle control spent significantly less time in the chamber previously paired with naloxone (FIG. A). By contrast, mice treated with Compound 1 (0.1 mg/kg; i.p) displayed significantly less CPA (FIG. B). Thus, the results indicate that Compound 1 attenuates both the physical and aversive components of opioid withdrawal.

### Compound 1 Reduces Opioid Withdrawal Aversion

Opioid withdrawal is aversive and manifests as physical signs and behaviours. To assess aversion, conditioned place avoidance (CPA) was used. CPA is a well-established behavioral test in which rodents avoid or spend less time in a chamber they previously experienced naloxone precipitated opioid withdrawal.

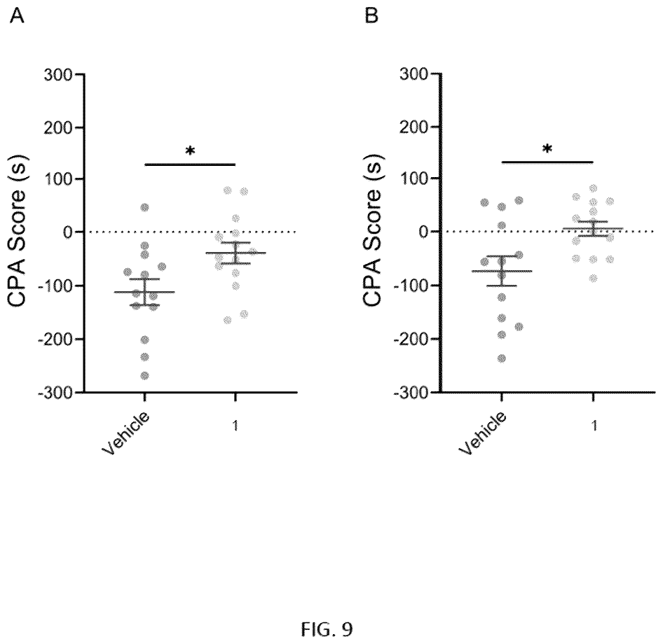

To examine withdrawal aversion, the CPA test was used wherein morphine-dependent mice underwent naloxone-precipitated withdrawal while confined to one side of a two-chamber place conditioning apparatus; these mice displayed robust withdrawal behaviours during the 30 min conditioning session. When allowed free access to both chambers 1 day (FIG. 9A) and 7 days (FIG. 9B) after naloxone-precipitated withdrawal, morphine-dependent mice administered vehicle control spent significantly less time in the chamber previously paired with naloxone. By contrast, mice treated with Compound 1 (0.1 mg/kg; i.p) displayed significantly less CPA. Thus, the results indicate that Compound 1 attenuates both the physical and aversive components of opioid withdrawal.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A compound having formula (I): 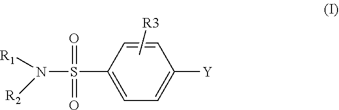
or a pharmaceutically acceptable salt thereof, wherein:
R1 and R2 are independently C₁ -C₄ linear alkyl, or R1 and R2 are alkyl groups taken together with the N to form a 4 to 8 membered cyclic group;
R3 is H, or one or more substituents independently selected from —R, —OH, —OR, —X, —CX₃, −NR′R″, C₄ -C₈ cyclic group or 5 to 7 membered heterocyclic group, wherein R is C₁-C₆ alkyl, X is F or Cl, R′ and R″ are independently H or C₁-C₆ alkyl;
Y is:
an unsaturated heterocyclic group having 1-3 N atoms, Optionally substituted with one or more substituents selected from —R, —OR, —COOH, —COOR, —NR′R″, wherein R is C₁ -C₆ alkyl, R′ and R″ are independently H or C₁ -C₆ alkyl or R′ and R″ are alkyl groups taken together with a heteroatom to form a 5 to 7 membered heterocyclic group,
or
a group having the following formula: 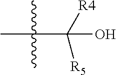
wherein R4 and R5 are independently selected from C₁-C₆ haloalkyl, wherein halogen is independently selected from F or Cl, aryl or heterocycle, wherein Y is other than:

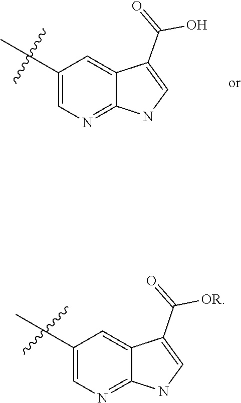

2. The compound of claim 1, wherein the unsaturated heterocyclic group is: 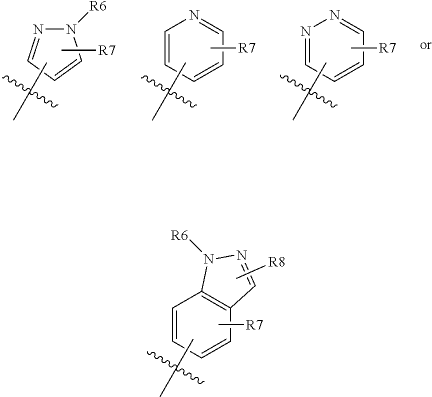
wherein:
R6 is H or alkyl, R7 is H or one or more substituents independently selected from —R, —OR, —NR′R″, X or CN, 5 to 7 membered heterocyclic group, wherein R is C1-C6 alkyl, X is F or Cl, and R′ and R″ are independently selected from H or alkyl, or R′ and R″ taken together with the N form a 5-6 membered cycle or heterocycle, and
R8 is selected from R, NR′R″, wherein R is C1-C6 alkyl, R′ and R″ are independently H or C1-C6 alkyl.

3. The compound of claim 1, which is: 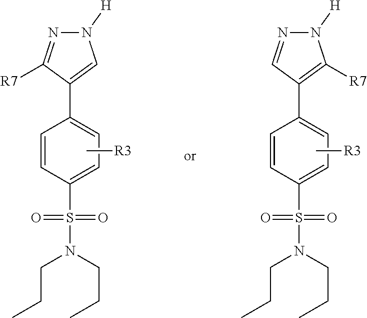
wherein, R3 as defined in claim 1, and
7 is H or one or more substituents independently selected from —R, —OR, —NR′R″, X or CN, 5 to 7 membered heterocyclic group, wherein R is C1-C6 alkyl, X is F or Cl, and R′ and R″ are independently selected from H or alkyl, or R′ and R″ taken together with the N form a 5-6 membered cycle or heterocycle.

4. The compound of claim 1, which is:

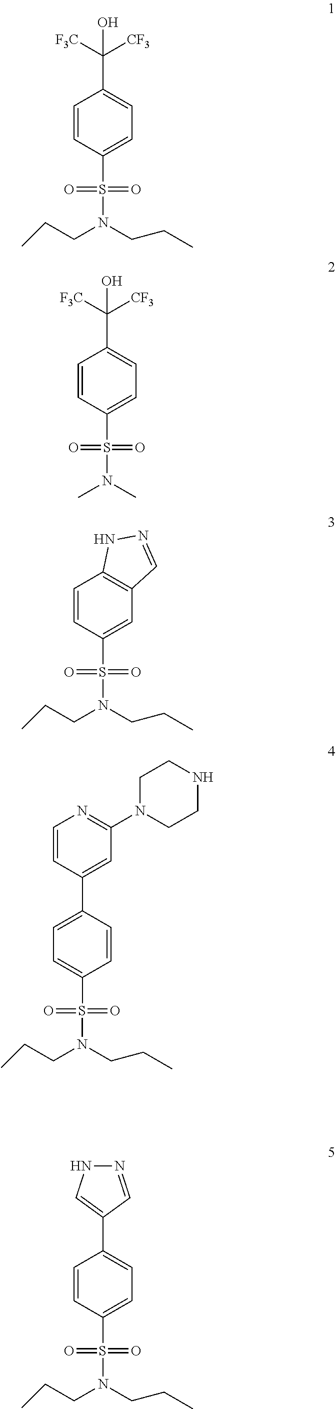

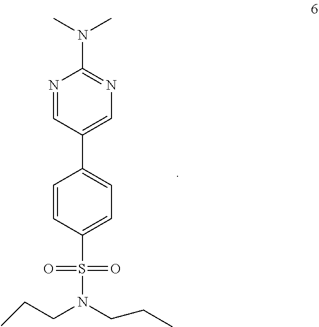 [SMILES: CCCN(CCC)S(=O)(=O)c1ccc(-c2cnc(N(C)C)nc2)cc1]

5. A pharmaceutical composition comprising a compound as defined in claim 1 and a pharmaceutically acceptable carrier.

6. A method of treating or ameliorating opioid withdrawal syndrome, or reducing risk of relapse to drug seeking in a subject, the method comprising administering to the subject an effective amount of the pharmaceutical composition as defined in claim 5.

7. The method according to claim 6, wherein the pharmaceutical composition is formulated for topical administration, administration with a transdermal patch, oral administration, or administration by injection.

8.-12. (canceled)

13. The method according to claim 7, wherein the composition is in the form of a lotion, a cream, a gel, or a viscous liquid.

14. The method according to claim 13, wherein the composition further comprises one or more of a skin penetration enhancer, an emollient, an emulsifying agent, a water miscible solvent, an alcohol, and mixtures thereof.

15.-25. (canceled)

26. A pharmaceutical composition comprising a compound as defined in claim 4 and a pharmaceutically acceptable carrier.

27. A method of treating or ameliorating opioid withdrawal syndrome, or reducing risk of relapse to drug seeking in a subject, the method comprising administering to the subject an effective amount of the pharmaceutical composition as defined in claim 26.

28. The method according to claim 27, wherein the pharmaceutical composition is formulated for topical administration, oral administration, or administration by injection.

29. The method according to claim 27, wherein the composition is in the form of a lotion, a cream, a gel, or a viscous liquid.

30. The method according to claim 29, wherein the composition further comprises one or more of a skin penetration enhancer, an emollient, an emulsifying agent, a water miscible solvent, an alcohol, and mixtures thereof.

31. A compound of formula:

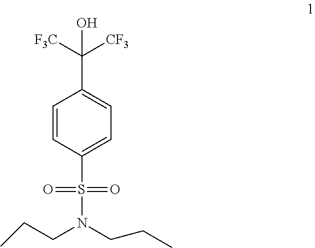 [SMILES: CCCN(CCC)S(=O)(=O)c1ccc(C(O)(C(F)(F)F)C(F)(F)F)cc1]

32. A pharmaceutical composition comprising the compound of claim 31, and a pharmaceutically acceptable carrier.

33. A method of treating or ameliorating opioid withdrawal syndrome, or reducing risk of relapse to drug seeking in a subject, comprising administering to the subject an effective amount of the pharmaceutical composition of claim 32.
